(19) United States
(12) Patent Application Publication
(10) Pub. No.: US 2023/0399318 A1
(43) Pub. Date: Dec. 14, 2023

(54) RENEWABLE OILS: COMPOSITION, PROCESS OF MAKING AND FORMULATION

(71) Applicant: RiKarbon, Inc, Newark, DE (US)

(72) Inventor: Basudeb Saha, Newark, DE (US)

(73) Assignee: RiKarbon, Inc, Newark, DE (US)

(21) Appl. No.: 18/033,583

(22) PCT Filed: Oct. 26, 2021
(86) PCT No.: PCT/US2021/056553

§ 371 Date: Apr. 25, 2023

Related U.S. Application Data
(60) Provisional application No. 63/105,404, filed on Oct. 26, 2020.

(51) Int. Cl. C07D 407/14; C07D 407/06; C07C 9/22; A61Q 19/00; A61K 8/92

(52) U.S. Cl. CPC C07D 407/14; C07D 407/06; C07C 9/22; A61Q 19/00; A61K 8/92; A61K 2800/80

(57) ABSTRACT

This invention relates to processes for making bio-based renewable oils from non-food biomass sources. Such renewable oils are used for cosmetics formulations. More specifically, disclosed are processes of preparation of furan-based compounds of the formula: (R₁-A)ₐ—CH(R₂)—CH₂—CH(R₃)(A-R1)_{b} wherein: a and b are independently 1 or 2 and 0 or 1; A is: independently an unsaturated, a partially hydrogenated, or a fully hydrogenated saturated fur an ring; —(CH₂)₄—; a saturated fur an ring-opened moiety containing a hydroxyl or a ketonic group: —CH₂—CH₂—CH₂—CH₂—OH or —CH₂—CH₂—CH₂—CH═O; or a partially saturated furan-ring opened moiety containing a hydroxyl or a ketonic group: —CH₂—CH₂—CH═CH—OH or —CH₂—CH═CH—CH═O where the position of the double bond is anywhere within the chain; R₁, R₂, and R₃ are: independently selected from H, a furan ring, a tetrahydrofuran ring and alkyl groups having carbon atoms of 1 to 18; and the total carbon content of the compound is from 10 to 40.

### CROSS REFERENCE TO RELATED APPLICATIONS

This application is based on U.S. Provisional Patent Application No.63/105,404, filed on Oct. 26, 2020. The priority of the foregoing applications are hereby claimed and their disclosures incorporated herein by reference in their entirety.

### STATEMENT OF FEDERALLY SPONSORED RESEARCH

This invention was made with a support grant from the Government of the United States under the DOE Grant No. DE-SC0018789 from the Department of Energy. The Government of the United States has certain rights in the present invention.

### FIELD OF INVENTION

This invention relates to efficient catalytic processes for making up to 100% bio-based, silicon-free, renewable oils from raw-materials that are sourced from non-food biomass, inedible oil seeds, natural oils. In the present invention, such renewable oils are used for cosmetics formulations. The performance of these renewable oils in cosmetic products formulation, for example, as emollients and their characteristic and sensory profiles are disclosed.

### BACKGROUND

Different oils are used as emollients and base-oils in cosmetic product formulations. Emollient, also referred to as cosmetic oil, is used in various cosmetics to easily solubilize emulsifiers in water-oil emulsions, to provide an aesthetic feel, and to provide a desired efficacy, in terms of its spreadability, and a feel for dry, shiny, silky and talc. The chemistry of emollient, and their application in cosmetics formulation for various desired properties have evolved over the years. One of the noticeable changes is the widespread use of cyclic silicones, also referred to as cyclosiloxanes. While these cyclic compounds, with different degree of volatility profile, do serve most needs of the cosmetic products formulation, they have been highly scrutinized in recent years for their safety and bio-accumulation concerns, and in fact, the use of these cyclic silicones has been restricted in most wash-off cosmetics. In addition, generation X and generation Z consumers' preference for natural, non-silicon clean beauty products has increased over the last decade. Recent consumer review statistics indicate that 50% consumers of all age groups avoid silicones in cosmetic products. As a result, alternative non-silicon ingredients such as linear and branched hydrocarbons such as isododecane, combination of different hydrocarbon mixtures, squalane, isosqualane, pentadecane, hexadecane; and esters such as neopentylglycol diheptanoate, PEG/PPG-8/3 Laurate, combination of esters such as PPG-3 isostearyl methyl ether, PPG-3 benzyl ether myristate, PPG-3 benzyl ether ethyl hexanoate, isoamyl cocoate, and diethylhexyl carbonate and others have been looked into by cosmetics manufacturers and formulators. However, most of these alternative ingredients are not derived from bio-based raw materials, and especially not from non-food, and sustainable, typically waste raw materials. It has been shown that most alternative ingredients do not perform as well as cyclic silicones.

### SUMMARY OF THE INVENTION

In general, this invention relates to: (1) the synthesis of a range of emollient and base oil compounds and compositions that possess a different molecular architecture, with carbon numbers ranging between C₁₀-C₄₀; and (2) a process of making such emollients and base oils. All compounds and compositions contain up to 100% bio-based carbon, They were synthesized from commercially procured raw materials that were derived from biomass, seed oils, and/or natural oils. In some embodiments, up to 100% carbon of these raw materials comes from sustainably sourced, typically waste feed-stocks.

In one embodiment, this invention relates to a compound having the following formula:

(R₁−A)ₐ—CH(R₂)—CH₂—CH(R₃)(A−R1)_{b}
wherein:
(i) each of a is independently 1 or 2;
(ii) each of b is independently 0 or 1;
(iii) A is independently
   (a) an unsaturated furan ring,
   (b) a partially hydrogenated furan ring,
   (c) a fully hydrogenated saturated furan ring,
   (d) a saturated furan ring-opened moiety containing a hydroxyl group or a ketonic group, that is, —CH₂—CH₂—CH₂—CH₂—OH or —CH₂—CH₂—CH₂—CH═O,
   (e) a partially saturated furan-ring opened moiety containing a hydroxyl group or a ketonic group, that is, —CH₂—CH₂—CH═CH—OH or —CH₂—CH═CH—CH═O where the position of the double bond is anywhere within the chain of the moiety,
   (f) —(CH₂)₄—;
(iv) R₁, R₂, and R₃ are independently selected from the group consisting of H, a furan ring, a tetrahydrofuran ring, and alkyl groups having carbon atoms of 1 to 18; and
(v) the total carbon content of the compound is in the range of 10 to 40.

In one embodiment, this invention relates to compound as recited above, wherein R₃ is a linear alkyl group.

In one embodiment, this invention relates to a compound as recited above, the compound having one of the following structures:

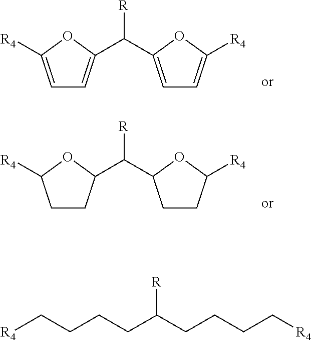

wherein R and R₄ are independently selected from the group consisting of H, a furan ring, a tetrahydrofuran ring, and alkyl groups having carbon atoms of 1 to 18.

In one embodiment, this invention relates to a compound as recited above, the compound having one of the following structures:

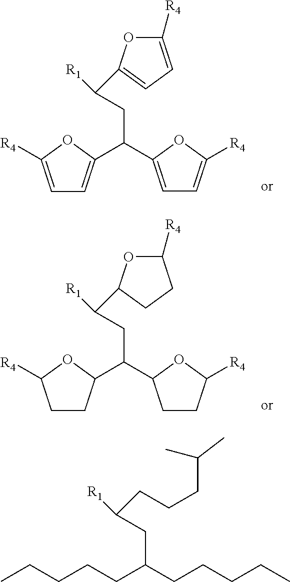

wherein R₁ and R₄ are independently selected from the group consisting of H, a furan ring, a tetrahydrofuran ring, and alkyl groups having carbon atoms of 1 to 18.

In one embodiment, this invention relates to a compound as recited above, characterized by having a bio-based carbon content in the range of 30 to 100%, according to ASTM D6866 method.

In one embodiment, this invention relates to a method of making a compound having the following formula:

(R₁−A)ₐ—CH(R₂)—CH₂—CH(R₃)(A−R1)_{b}
wherein:
(i) each of a is independently 1 or 2;
(ii) each of b is independently 0 or 1;
(iii) A is independently
   (a) an unsaturated furan ring,
   (b) a partially hydrogenated furan ring,
   (c) a fully hydrogenated saturated furan ring,
   (d) a saturated furan ring-opened moiety containing a hydroxyl group or a ketonic group, that is, —CH₂—CH₂—CH₂—CH₂—OH or —CH₂—CH₂—CH₂—CH═O,
   (e) a partially saturated furan-ring opened moiety containing a hydroxyl group or a ketonic group, that is, —CH₂—CH₂—CH═CH—OH or —CH₂—CH═CH—CH═O where the position of the double bond is anywhere within the chain of the moiety, or
   (f) —(CH₂)₄—
(iv) R₁, R₂, and R₃ are independently selected from the group consisting of H, a furan ring, a tetrahydrofuran ring, and alkyl groups having carbon atoms of 1 to 18; and
(v) the total carbon content of the compound is in the range of 10 to 40;
the method comprising:
(A) performing an acid catalyzed condensation reaction of:
   (I) a first component, comprising one or more of:
   a 2-alkylfuran having the formula R₁-A, wherein R₁ is independently selected from the alkyl groups having carbon number of 1 to 18, and A is a furan ring, with
   (II) a second component, comprising one or more of:
      (a) an aldehyde or a ketone having the formula of O═C(R₂)—R₅ wherein R₅ is independently selected from the alkyl groups having carbon number of 1 to 17, and R₂ is independently selected from the group consisting of H and alkyl groups having carbon number of 1 to 18,
      (b) an aldehyde having the formula of O═CH—R wherein R is a furan ring or a tetrahydrofuran ring having carbon number of 4,
      (c) an aldehyde or a ketone having the formula of O═C(R₂)—CH═CH—R₃ wherein R₂ is independently selected from the group consisting of H and alkyl groups having carbon number of 1 to 17, and R₃ is independently selected from the alkyl groups having carbon number of 1 to 17,
   wherein at least one of the first component and the second component is at least partially bio-derived from a renewable carbon feedstock,
(B) selectively hydrogenating the condensation compound from Step (A) in the presence of a hydrogenation catalyst to obtain a saturated furan product, referred hereto hydrogenated saturated condensation compound or as RKsat-n,
(C) performing selective hydrodeoxygenation of:
   (c1) the condensation compound in Step (A), or
   (c2) the hydrogenated saturated condensation compound of Step (B),
   in the presence of a hydrodeoxygenation catalyst to obtain a compound referred hereto as RKBA-n.

In one embodiment, this invention relates to a method as recited above, wherein the renewable carbon feedstock is selected from the group consisting of biomass, rapeseeds, palm kernel, natural oils, waste cooking oils, castor seed, corn grain, soya bean grain, hard wood, soft wood, algae, natural coconut oil, palm oil, rapeseed oil, vegetable oil, corn oil, peanut oil, olive oil, canola oil, and sunflower oil. any waste cooking oils of one or more natural cooking oils and/or animal fats, combinations thereof, and mixtures thereof.

In one embodiment, this invention relates to a method as recited above, wherein R₃ is a linear alkyl group.

In one embodiment, this invention relates to a method as recited above, the compound having one of the following structures:

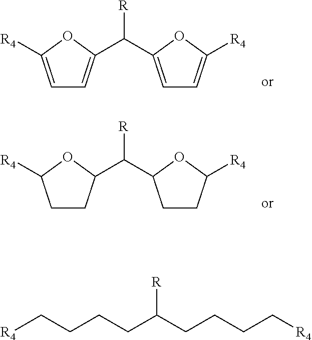

wherein R and R₄ are independently selected from the group consisting of H, a furan ring, a tetrahydrofuran ring, and alkyl groups having carbon atoms of 1 to 18.

In one embodiment, this invention relates to a method as recited above, the compound having one of the following structures:

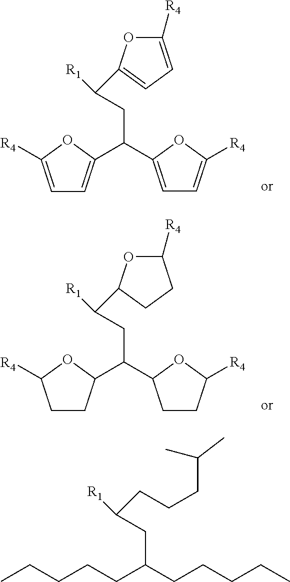

wherein R₁ and R₄ are independently selected from the group consisting of H, a furan ring, a tetrahydrofuran ring, and alkyl groups having carbon atoms of 1 to 18.

In one embodiment, this invention relates to a method as recited above, wherein the step of providing an aldehyde comprises at least one of:
(i) dehydrogenating of one or more biomass derived alcohols,
(ii) selective hydrogenation of one or more carboxylic acids from one or more natural oils or waste cooking oils,
(iii) dehydration of biomass derived C₅ sugars, or
(iv) pyrolysis of castor seed.

In one embodiment, this invention relates to a method as recited above, wherein the 2-alkylfuran has been prepared by a process, comprising:
(i) dehydration and hydrodeoxygenation of one or more C₅ sugars of biomass, or
(ii) furan acylation with one or more carboxylic acids or carboxylic acid anhydrides, followed by hydrodeoxygenation of the acylated products.

In one embodiment, this invention relates to a method as recited above, wherein the catalyst comprises one acidic catalyst selected from the group consisting of organic liquid acids, inorganic liquid acids, solid Bronsted acids, and combinations thereof.

In one embodiment, this invention relates to a method as recited above, wherein the acidic catalyst is selected from acidic resins, fluorinated resins, zeolites, phosphoric acid, phosphorous silica, orthophosphoric acid, HCl, H₂SO₄, methanesulfonic acid, p-toluene sulfonic acid, and combinations thereof.

In one embodiment, this invention relates to a method as recited above:
wherein the hydrogenation catalyst comprises one metal catalyst selected from the group consisting of Ir, Ni, Pd, Pt, Ru, Mb, Zn, Ti, V, Cr, Mn, Fe, Co, and combinations thereof and wherein said hydrogenation catalyst is supported on a support material selected from the group consisting of activated carbon, porous carbon, silica, polymeric hybrid material, and weakly acidic material, mixture of Ni/NiO, Ni/C, Pd/C, Ru/C, Ni/SiO2, Pd/SiO2, Ni on zeolite supports, Ni/ZSM5, WR Grace-Raney Ni 2800, WR Grace-Raney Ni 3200, and a mixture of Ni/NiO with Al2O3, ZrO2, Kieselguhr, Cr2O3 in different ratios.

In one embodiment, this invention relates to a method as recited above, wherein the hydrodeoxygenation catalyst comprises the catalyst consisting of a metal selected from a hydrogenation metal or a base metal, an acid site selected from a Lewis acid or a Bronsted acid, or a mixture thereof.

In one embodiment, this invention relates to a method as recited above, wherein the hydrodeoxygenation catalyst is:
(i) a bifunctional metal-acid catalyst co-existing with the metal and acid sites selected from the group consisting of Ni/ZSM5, Ni/zeolite, Ni/silica, Ni/Al2O3, Raney Ni 2800, Raney Ni 3200, Pd/ZSM5, Pd/zeolite, Pd/silica, Pd/Al2O3, a mixture of Ni/NiO with Al2O3, ZrO2, Kieselguhr, Cr2O3 with different ratios or a physically mixed catalyst consisting of a hydrogenation catalyst and an acid catalyst, or a supported metal-metal oxide catalyst of a general formula M¹MO wherein M¹=Ir, Ru, Ni, Co, Pd, or Rh and M=Re, Mo, W, Nb, Mn, V, Ce, Cr, Zn, Co, Y, or Al,
(ii) a physically-mixed catalyst comprising a hydrogenation catalyst and an acid catalyst, or
(iii) a supported metal-metal oxide catalyst of a general formula M¹MO,
wherein M¹=Ir, Ru, Ni, Co, Pd, or Rh; and
M=Re, Mo, W, Nb, Mn, V, Ce, Cr, Zn, Co, Y, or Al.

In one embodiment, this invention relates to a cosmetic composition, comprising:
(A) one or more compounds as recited in claims 1-5; and
(B) an effective amount of one or more additives selected from the group of pigment, fragrance, emulsifier, wetting agent, thickener, emollient, rheology modifier, viscosity modifier, gelling agent, antiperspirant agent, deodorant active, fatty acid salt, film former, anti-oxidant, humectant, opacifier, monohydric alcohol, polyhydric alcohol, fatty alcohol, preservative, pH modifier, a moisturizer, skin conditioner, stabilizing agent, proteins, skin lightening agents, skin-darkening agents, topical exfoliants, antioxidants, retinoids, refractive index enhancer, photo-stability enhancer, SPF improver, UV blocker, antibiotic agents, antiseptic agents, antifungal agents, anti-microbial agents, corticosteroid agents, anti-acne agents, and water.

In one embodiment, this invention relates to a cosmetic composition as recited above, wherein said one or more compounds as recited in (A) comprise 0.1% to 99% by weight of the cosmetic composition.

In one embodiment, this invention relates to a cosmetic composition as recited above, wherein the one or more compounds have a furan ring, a tetrahydrofuran ring, a partially hydrogenated furan ring, a partially saturated furan-ring opened moiety containing a hydroxyl or a ketonic group, that is, —CH₂—CH₂—CH═CH—OH or —CH₂—CH═CH—CH═O, where the position of the double bond is anywhere within the moiety, or —(CH₂)₄—.

In one embodiment, this invention relates to a cosmetic composition as recited above, wherein the one or more compounds have a branched molecular structure or a linear molecular structure.

In one embodiment, this invention relates to a cosmetic composition as recited above, wherein the one or more compounds have an ether oxygen atom linkage or no oxygen atom.

In one embodiment, this invention relates to skin care cosmetics composition, a decorative cosmetics composition, a hair-care cosmetic composition, or a personal care cosmetics composition comprising the cosmetic composition as recited above.

In one embodiment, this invention relates to cosmetic compositions as recited above, for using as or in the preparation of, emollients, lotions, water-in-oil emulsions, oil-in-water emulsions, creams, gels, flexible solids, skin care cosmetics, moisturizing preparations, ointments, oils, cleansers, make-up removers, night and day treatments, skin reparatives, facial sun defense, sunscreens, blush, liquid blush, transparent blush, perfecting primer, liquid soaps, color foundations, make-ups, concealing sticks, rouge, tanning creams, eye shadow creams, shampoo, body wash, facial cleanser, face mask, bubble bath, intimate wash, bath oil, cleansing milk, micellar water, cleansing wipes, hair mask, perfume, liquid soap, shaving soap, shaving foam, cleansing foam, day cream, anti-ageing cream, body milk, body lotion, body mousse, face serum, eye cream, sunscreen lotion, sun cream, face cream, after-shave lotion, pre-shaving cream, depilatory cream, skin-whitening gel, self-tanning cream, anti-acne gel, mascara, foundation, primer, concealer, blush, bronzer, blemish balm (bb) cream, eyeliner, night cream, eye brow gel, highlighter, lip stain, hand sanitizer, hair oil, nail varnish remover, conditioner, hair styling gel, hair styling cream, anti-frizz serum, scalp treatment, hair colorant, split end fluid, deodorant, antiperspirant, baby cream, insect repellent, hand cream, sunscreen gel, foot cream, exfoliator, body scrub, cellulite treatment, bar soap, cuticle cream, lip balm, hair treatment, eye shadow, bath additive, body mist, eau de toilette, mouthwash, toothpaste, lubricating gel, moisturizer, serum, toner, aqua sorbet, cream gel, styling mousse, dry shampoo, lip stick, lip gloss, hydro-alcoholic gel, body oil, shower milk, illuminator, lip crayon, hair spray, combing cream, sunblock, cosmetic product that colors the skin, cosmetic product that lightens the skin, cosmetic products that repairs the skin, cosmetic product that moisturizes the skin, cosmetic product that smooth the skin, cosmetic product that conditions the skin, cosmetic products that protects the skin, cosmetic product that cleans the skin, cosmetic product that rejuvenates the skin, cosmetic product that prevents the loss of moisture, and cosmetic product that reverses damages of the skin.

In one embodiment, this invention relates to a process for preparing a cosmetic composition as recited in claim 24;
said process comprising the steps of
(I) providing:
   (A) at least one compound according to claim 1, and
   (B) an effective amount of one or more additives selected from the group consisting of pigment, fragrance, emulsifier, wetting agent, thickener, emollient, rheology modifier, viscosity modifier, gelling agent, antiperspirant agent, deodorant active, fatty acid salt, film former, anti-oxidant, humectant, opacifier, monohydric alcohol, polyhydric alcohol, fatty alcohol, preservative, pH modifier, a moisturizer, skin conditioner, stabilizing agent, proteins, skin lightening agents, topical exfoliants, antioxidants, retinoids, refractive index enhancer, photo-stability enhancer, SPF improver, UV blocker, antibiotic agents, antiseptic agents, antifungal agents, corticosteroid agents, anti-acne agents, water and mixtures thereof; and
(II) physically or chemically mixing or blending the ingredients in (A) and (B).

### BRIEF DESCRIPTION OF DRAWINGS

FIG. 1 depicts the molecular structures of the emollient compounds of the present invention.

FIG. 2 depicts the GC profile of the compound RKunsat-17.

FIG. 3 depicts the GC profile of the compound RKunsat-15.

FIG. 4 depicts the GC profile of the compound RKunsat-22.

FIG. 5 depicts the GC profile of the compound RKsat-17.

FIG. 6 depicts the GC profile of the compound RKsat-22.

FIG. 7 depicts the GC profile of the compound RKsat-15.

FIG. 8 depicts the GC profile of the compound RKBA-17.

FIG. 9 depicts the GC profile of the compound RKBA-22.

FIG. 10 depicts the GC profile of the compound RKBA-15.

FIG. 11 depicts a standard spider diagram showing sensory profiles for RKBA-17 and RKBA-22.

### DETAILED DESCRIPTION OF THE INVENTION

As used herein, the term “biomass-derived” is used interchangeably with “biologically-derived”, “bio-derived” or “bio-based” and refers to compounds that are obtained from renewable resources such as plants. Such compounds contain substantially renewable carbon or only substantially renewable carbon. Also, such compounds contain no fossil fuel-based carbon or petroleum-based carbon or a very minimal amount of fossil fuel-based or petroleum-based carbon.

Assessment of the renewable resource-based carbon in a material can be performed through standard test methods. Using radiocarbon and isotope ratio mass spectrometry analysis, the bio-based content of materials can be determined, using ASTM-D6866, a standard method established by ASTM International, formally known as the American Society for Testing and Materials.

As used herein, the “bio-based content” is determined in accordance with ASTM-D6866 and is built on the same concepts as radiocarbon dating, but without use of the age equations. The analysis is performed by deriving a ratio of the amount of radiocarbon (14C) in an unknown sample to that of a modern reference standard. The ratio is reported as a percentage with the units “pMC” (percent mod- ern carbon) with modern or present defined as 1950. If the material being analyzed is a mixture of present day radiocarbon and fossil carbon (containing no radiocarbon), then the pMC value obtained correlates directly to the amount of biomass material present in the sample.

Combining fossil carbon with present-day carbon into a material will result in a dilution of the present-day pMC content. By presuming 107.5 pMC represents present-day biomass materials and 0 pMC represents petroleum derivatives, the measured pMC value for that material will reflect the proportions of the two component types . A material derived 100% from present day plant/tree would give a radiocarbon signature near 107.5 pMC. If that material was diluted with 50% petroleum derivatives, it would give a radiocarbon signature near 54 pMC.

A bio-mass content result is derived by assigning 100% equal to 107.5 pMC and 0% equal to 0 pMC. In this regard, a sample measuring 99 pMC will give an equivalent bio-based content result of 93%.

Assessment of the biodegradability of a material, such as of compounds of formula (I), base oils, or cosmetic compositions of the present disclosure, can be performed through standard test methods, such as those developed by the Organization for Economic Cooperation and Development (OECD), the Coordinating European Council (CEC), and the ASTM, such as, OECD 301B (the Modified Strum test), ASTM D-5864, and CEC L-33-A-934. Both OECD 301B and ASTM D-5864 measure ready biodegradability, defined as the conversion of 60% of the material to CO₂ within a ten-day window following the onset of biodegradation, which must occur within 28 days of test initiation. In contrast, the CEC method tests the overall biodegradability of hydrocarbon compounds and requires 80% or greater biodegradability as measured by the infrared absorbance of extractable lipophilic compounds.

By “cosmetic composition” of the invention is meant a cosmetic formulation or a cosmetic formulation precursor that comprises at least one compound of the present invention or at least one compound of the present invention made by the processes of the present invention.

By “cosmetic formulation” is meant a formulation that comprises at least one compound of the present invention, including the unsaturated compounds, the saturated compounds, and the branched compounds. The cosmetic formulation can be used for skin care, as decorative cosmetics, or personal care cosmetics.

By “cosmetic formulation precursor” is meant a formulation that may undergo additional modification, for example, by addition of one or more ingredients, or physical or chemical treatment to render it into a “cosmetic formulation” for use in skin care, decorative cosmetics, or personal care cosmetics.

As used herein, the terms “emollients,” “cosmetic emollients,” and “cosmetic oils” refer to any substance used to formulate end-used cosmetics that allow formation of emulsions in the presence of emulsifiers, water, pigments, additives and other functions and serve as a skin softener and moisturizer by increasing the ability of the skin to hold water, providing the skin with a layer of oil to prevent water loss, and lubricating the skin. In general, an emollient exhibits one or more characteristics, such as moisturizing, spreadability, glossiness, firmness, stringiness, stickiness, absorbency, miscibility with emulsifier and water, pignut wettability, thermal stability, oxidation stability, stability at skin pH, and lubricity.

As used herein, the terms “cosmetic solvent,” and “solvent in sunscreen formulation,” refer to any substance used to formulate end-used cosmetic compositions or sunscreen products that allow high solubility of sunscreen active ingredients in the formulation of end-use sunscreen products.

As used herein, a “condensation” reaction refers to a chemical reaction in which two molecules combine to form larger molecule while producing a small molecule, such as H₂O, as a byproduct.

As used herein, a “hydrogenation” reaction refers to a chemical reaction between molecular hydrogen and another compound, typically, in the presence of a catalyst to reduce or saturate organic compounds.

As used herein, a “hydrodeoxygenation” or “HDO” reaction refers to a chemical reaction whereby a carbon-oxygen single bond is cleaved or undergoes lysis (cleavage of a C—O bond) by hydrogen, typically in the presence of a catalyst. “HDO” is a process for removing oxygen from a compound.

The term “kinematic viscosity” is used herein to refer to a fluid's inherent resistance to flow when no external force other than gravity is acting on the fluid. “Kinematic viscosity” is measured as the ratio of absolute (or dynamic) viscosity to density.

The term “pour point” as used herein refers to the temperature below which a liquid loses its flow characteristics.

The term “saturated” as used herein refers to an organic molecule containing the greatest number of hydrogen atoms and no carbon-carbon double or triple bonds.

A “saturated” furan ring refers to a furan ring (a five membered ring containing four carbon atoms and an oxygen atom) with the greatest number of carbon-carbon single bonds, e.g., tetrahydrofuran. An “unsaturated” furan ring refers to a furan ring with the maximum number of carbon-carbon double bonds, and is interchangeably used herein with “condensed furan.” A “partially saturated” furan ring contains at least one carbon-carbon double bond but contains less than the maximum number of carbon-carbon double bonds, e.g., dihydrofuran.

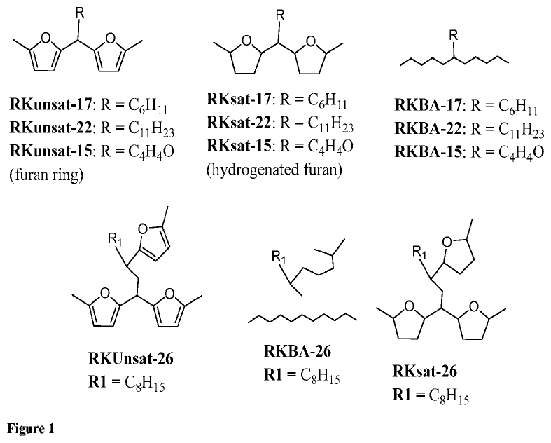

The present invention relates to the composition of compounds for cosmetic products, their methods of preparation using catalysts, and their formulation recipes for cosmetic applications. General molecular formulae of these emollients containing furan rings, tetrahydrofuran rings and branched hydrocarbons without any oxygen include RKunsat-n, RKsat-n and RKBA-n, respectively, where n represents total number carbon atoms in the molecular structures of compounds. Specifically, it discloses compositions of new compounds (e.g., RKunsat-17, RKsat-17, RKBA-17, RKunsat-26, RKBA-26; FIG. 1), methods of preparation of all compounds, and cosmetic formulation of compounds as emollients. The syntheses involved two-step catalytic reactions of commercially procured bio-based raw materials, derived either from non-food biomass and/or inedible oil seeds/natural oils, followed by distillation and purification. The catalysts were prepared in-house or procured from commercial sources. The yield, purity, and selectivity of each compound were determined using chromatography and mass spectrometry techniques.

The general formula of the compounds of the present invention is provided below:

(R₁−A)ₐ—CH(R₂)—CH₂—CH(R₃)(A−R1)_{b}
wherein:
(i) each of a is independently 1 or 2;
(ii) each of b is independently 0 or 1;
(iii) A is independently an unsaturated furan ring, a partially hydrogenated furan ring, a fully hydrogenated saturated furan ring, a saturated furan ring-opened moiety containing a hydroxyl or a ketonic group, i.e., —CH₂—CH₂—CH₂—CH₂—OH or —CH₂—CH₂—CH₂—CH═O, a partially saturated furan-ring opened moiety containing a hydroxyl or a ketonic group, i.e., —CH₂—CH₂—CH═CH—OH or —CH₂—CH═CH—CH═O where the position of the double bond is anywhere within the chain of the moiety, or —(CH₂)₄—;
(iv) R₁, R₂, and R₃ are independently selected from the group consisting of H, a furan ring, a tetrahydrofuran ring and alkyl groups having carbon atoms of 1 to 18; and
(v) the total carbon content of the compound is in the range of 10 to 40. characterized by having a bio-based carbon content in the range of 20 to 100%, according to ASTM D6866 method. Stated differently, the bio-based carbon content can be any number below, or within a range defined by any two numbers below, including the endpoints of such a range:
20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66 ,67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 07, 08, 99, and 100.

Renewable carbon feedstocks from which the above compounds are prepared include biomass, rapeseeds, palm kernel, natural oils, waste cooking oils, castor seed, corn grain, soya bean grain, any kind of hard wood, any kind of soft wood, algae, any suitable natural cooking oils including, but not limited, to coconut oil, palm oil, rapeseed oil, vegetable oil, corn oil, peanut oil, olive oil, canola oil, and sunflower oil. any waste cooking oils of one or more natural cooking oils and/or animal fats.

In one embodiment, the cosmetic compositions of the present invention do not contain silicon compounds.

### Process of Making a Compound Represented by Formula (I)

The invention disclosed herein include processes for the preparation of a compound as represented by the formula (I) from one or more bio-derived reactants, the compounds of formula (I), and their use as emollients in cosmetic compositions. In an embodiment, the compounds of formula (I) is an unsaturated furan (unsat) compound, a saturated furan (sat) compound, or a branched alkane (BA) compound.

In an aspect of the invention, the compound is represented by the following formula:

(R₁−A)ₐ—CH(R₂)—CH₂—CH(R₃)(A−R1)_{b} Formula (I)
wherein, each of a is independently 1 or 2; and each of b is independently 0 or 1.

In one embodiment, each A is independently a saturated furan ring, a partially saturated furan ring, an unsaturated furan ring, or a saturated furan ring-opened moiety —(CH₂)₄—. In another embodiment, each A is independently a saturated furan ring, an unsaturated furan ring, or saturated furan ring-opened moiety —(CH₂)₄—. In yet another embodiment, each A is independently a saturated furan ring, a partially saturated furan ring, or an unsaturated furan ring. In some embodiment, each A is independently a saturated furan ring, or an unsaturated furan ring.

In the compound of Formula (I), R₁, R₂, and R₃ can be independently chosen from among H (hydrogen), a furan ring, a tetrahydrofuran ring, and alkyl groups having 1 to 18 carbon atoms. In one embodiment, at least one of R₁, R₂, and R₃ is not H (hydrogen), and the total carbon content of the compound of formula (I) is in the range of 10 to 40 (meaning that the compound contains a total of from 10 to 40 carbon atoms). In one embodiment, at least one of R₁, R₂, and R₃ is H (hydrogen), and the total carbon content of the compound of formula (I) is in the range of 10 to 40 (meaning that the compound contains a total of from 10 to 40 carbon atoms). In an embodiment, both R₂ and R₃ of compounds of Formula (I) may be H.

As used herein the alkyl groups can be substituted or unsubstituted, cyclic or acyclic, or branched or unbranched or a combination thereof. Suitable examples of alkyl groups include, but are not limited to, butyl, pentyl, hexyl, heptyl, octyl, dodecyl, octadecanyl, cyclopentyl, and cyclohexyl.

In an embodiment, R₁, R₂, and R₃ are different. In yet another embodiment R₁, R₂, and R₃ are the same. In one embodiment, R₁, R₂, and R₃ are independently chosen from branched alkyl groups having 3-18 carbon atoms, preferably from acyclic branched alkyl groups having 3-11 carbon atoms, and most preferably from acyclic branched alkyl groups having 4-8 carbon atoms, provided that in total the compound contains from 10 to 40 carbon atoms.

In yet another embodiment, R₁, R₂, or R₃ may have the formula: —(CHR₆)—CH₂R₇). In such embodiments, R₆ and R₇ can be independently H or a linear or branched alkyl group having 2 to 18 carbon atoms, preferably a branched alkyl group having 2-11 carbon atoms, and most preferably an acyclic branched alkyl group having 3-11 carbon atoms, provided that in total the compound contains from 10 to 40 carbon atoms. Suitable examples of R₆, R₇ include, but are not limited to, methyl, butyl, hexyl, dodecyl, cyclopentyl, and cyclohexyl. Suitable examples of R₁, R₂, and R₃ include, but are not limited to, butyl, pentyl, hexyl, heptyl, octyl, dodecyl, octadecanyl, cyclopentyl, and cyclohexyl.

In an embodiment, one of R₁, R₂, and R₃ is hydrogen. In another embodiment, all of R₁, R₂, and R₃ are hydrogen. In yet another embodiment, R₁, R₂, and R₃ may be independently chosen from among branched alkyl groups having 1-9 carbon atoms, preferably 1-8 carbon atoms, and most preferably 1-6 carbon atoms, provided that in total the compound contains from 10 to 40 carbon atoms. Suitable examples of R₁, R₂, and R₃ include, but are not limited to, methyl, butyl, hexyl, dodecyl, cyclopentyl, and cyclohexyl.

In one embodiment, one of R₁, R₂, and R₃ is hydrogen. In another embodiment, both R₂ and R₃ are hydrogen. In yet another embodiment, at least one of R₁, R₂, and R₃ is an alkyl chain having 2-8 carbon atoms, preferably 2-6 carbon atoms, and most preferably 4-6 carbon atoms, provided that in total the compound contains 10 to 40 carbon atoms.

In an embodiment, at least one of one of R₁, R₂, and R₃ is a branched alkyl group, having one or more branches. The one or more branches can have any suitable number of carbon atoms, with at least one of the branches having 1-18 carbon atoms, and preferably 1-10 carbon atoms. The branched alkyl group may, for example, contain a total of 3-18 carbon atoms or 3-11 carbon atoms. Suitable examples of branched alkyl groups, having one or more branches include, but are not limited to, methylpropyl, methylbutyl, methyldodecyl, ethylpropyl, ethyloctyl, and cyclopentyheptyl.

In an aspect, the compound of Formula (I) has one of the following structures:

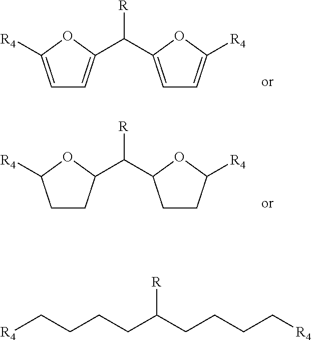

wherein R and R₄ are independently H, a furan ring, a tetrahydrofuran ring and alkyl groups having carbon atoms of 1 to 18.

As used above, in one embodiment, the alkyl groups can be substituted or unsubstituted, cyclic or acyclic, or branched or unbranched or a combination thereof. Suitable examples of alkyl groups include, but are not limited to, butyl, pentyl, hexyl, heptyl, octyl, dodecyl, octadecanyl, cyclopentyl, and cyclohexyl. In another embodiment, R and R4 may be independently chosen from among hydrogen or a substituted or an unsubstituted, a cyclic or an acyclic, a branched or an unbranched alkyl group having 1 to 18 carbon atoms, preferably 2-12 carbon atoms, and most preferably 4-10 carbon atoms, provided that in total the compound contains from 10 to 40 carbon atoms.

In an aspect, the compound of Formula (I) has one of the following structures:

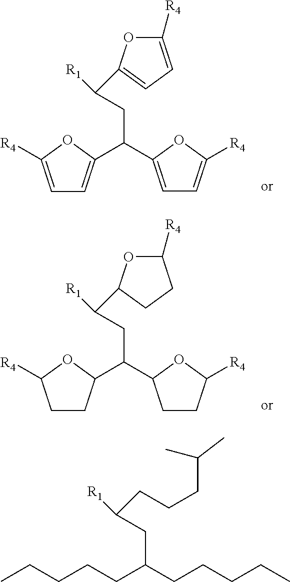

wherein R₁ and R₄ are independently H, a furan ring, a tetrahydrofuran ring or alkyl groups having carbon atoms of 1 to 18.

As used above, in one embodiment, the alkyl groups can be substituted or unsubstituted, cyclic or acyclic, or branched or unbranched or a combination thereof. Suitable examples of alkyl groups include, but are not limited to, butyl, pentyl, hexyl, heptyl, octyl, dodecyl, octadecanyl, cyclopentyl, and cyclohexyl. In another embodiment, R1 and R4 may be independently chosen from among hydrogen or a substituted or an unsubstituted, a cyclic or an acyclic, a branched or an unbranched alkyl group having 1 to 18 carbon atoms, preferably 2-12 carbon atoms, and most preferably 4-10 carbon atoms, provided that in total the compound contains from 10 to 40 carbon atoms.

In one embodiment, the compound of formula (I) is an unsaturated furan (unsat) compound. In another embodiment, the compound of formula (I) is a branched alkane (BA) compound. In yet another embodiment, the compound of formula (I) is a saturated furan (sat) compound In another embodiment, the compound of formula (I) is a partially saturated furan (Psat) or a mixture of one or more of unsat, and Psat. Exemplary unsat compounds include, but are not limited to: 5,5′-(heptane-1,1-diyl)bis(2-methylfuran), 5,5(furan-2-ylmethylene)bis(2-methylfuran), 5,5′-(dodecane-1,1-diyl)bis(2-methylfuran), 5,5′,5″-(undecane-1,1,3-triyl)tris(2-methylfuran).

Exemplary sat compounds include, but are not limited to:5,5′-(heptane-1,1-diyl)bis(2-methyltetra-hydrofuran), 5,5′-(do decane-1,1-diyl)bis(2-methyltetrahydrofuran), 5,5″((tetrahydrofuran-2-yl)methylenebis(2-methylenetetrahy drofuran).

Exemplary BA compounds include, but are not limited to: 6-pentylheptadecane, 6-pentyldodecane, 6-butylundecane, 7-(4-methylpentyl)-6-pentylpentadecane.

Referring back to the process of making a compound as represented by formula (I), the process includes providing one or more of an aldehyde, a dialdehyde, an enal or a ketone and one or more 2-alkylfurans, where at least one of the 2-alkylfurans, the aldehyde, the dialdehyde, the enal or the ketone is bio-derived from a renewable source.

Any suitable aldehyde (R₄R₅CO), dialdehyde ((CR₄R₅)n(CHO)₂), enal (CHR₂═CR₃—CHO), or ketone (R₄R₅CO) may be used. In the aldehyde (R₄R₅CO), at least one of R₄ or R₅ is hydrogen and at most one of R₄ or R₅ is an alkyl group having 1 to 18 carbon atoms or 2 to 8 carbon atoms. In the dialdehyde (CR₄R₅)n(CHO)₂, each R₄ and R₅ may be independently selected from the group consisting of H and alkyl groups having 1 to 18 carbon atoms or 2 to 8 carbon atoms, with n being an integer of 2-8. In the enal (CHR₂═CR₃—CHO), R₂ and R₃ independently may be H or an alkyl group having 1 to 18 or 2 to 8 carbon atoms. In the ketone (R₄R₅CO), R₄ and R₅ may be independently selected from the group consisting of alkyl groups having 1 to 8 carbon atoms. Any suitable one or more 2-alkylfurans (R₁-A) may be used, wherein R₁ is as defined hereinabove and A is the furan ring.

In an embodiment, the step of providing an aldehyde includes at least one of dehydrogenating biomass derived alcohols or selective hydrogenation of fatty acids from natural oils or waste cooking oils or pyrolysis of castor seed or dehydration of biomass derived sugars, e.g., xylose and hexose sugars. Suitable examples of biomass derived aldehydes include, but not limited to, furfural, 5-hdroxymethyfurfural, butanal, heptanal, dodecanal, trans-2-decenal. Suitable examples of biomass derived alcohols include, but are not limited to, ethanol, butanol, hexanol, undecanol and dodecanol. Such biomass derived alcohols may be derived from any suitable biomass including, but not limited to, corn grain, soya bean grain, any kind of hard wood, any kind of soft wood, and algae. Suitable examples of fatty acids include, but are not limited to lauric acid, heptanoic acid and steric acid. Such fatty acid may be derived from any suitable natural cooking oils including, but not limited, to coconut oil, palm oil, rapeseed oil, vegetable oil, corn oil, peanut oil, olive oil, canola oil, and sunflower oil. The fatty acids may also be derived from any waste cooking oils of one or more natural cooking oils and/or animal fats. The synthesis of aldehydes of different carbon length via dehydrogenation of biomass derived alcohols or selective hydrogenation of fatty acids from natural oils or WCO or dehydration of biomass derived xylose or hexose sugars is known in the art.

In another embodiment, the step of providing an enal includes the step of dimerization of an aldehyde.

In an embodiment, the 2-alkylfuran (or a mixture of 2-alkylfurans) may be prepared by a process comprising dehydration and hydrodeoxygenation of C5 sugars, e.g. xylose, of biomass such as corn grain, soya bean grain, any kind of hard wood, any kind of soft wood, algae and the like. In another embodiment, the 2-alkylfuran (or a mixture of 2-alkylfurans) may be prepared by a process comprising furan acylation with carboxylic acids or carboxylic acid anhydrides followed by hydrodeoxygenation of the acylated products. The synthesis of 2-alkylfurans of different carbon numbers via direct HDO of C₅ sugars of biomass, i.e., 2-methylfuran (2MF), or furan acylation with carboxylic acids (or their anhydrides) followed by HDO of the acylated products is known in the art.

The process of making a compound of formula (I) further includes condensation reaction of a 2-alkylfuran with an aldehyde, a dialdehyde, an enal or a ketone optionally in the presence of an acidic catalyst to form an unsaturated furan compound (unsat).

In an embodiment, the process of making a compound of formula (I) may also include hydrogenating the unsaturated furan compound in the presence of a hydrogenation catalyst to obtain a saturated furan compound (sat). Partial hydrogenation of the condensed furan compound may also be carried out, thereby forming a condensed partially saturated furan compound (containing one or more dihydrofuran rings).

### Catalytic Processes

The processes of the present invention, condensation, hydrogenation, and hydrodeoxygenation are catalytic processes.

Catalysts used in the processes of the present invention include metal-based catalysts such as at least one from iridium, nickel, palladium, platinum, rhodium, ruthenium, cobalt, and copper. The materials for hydrogenation reactions in metal catalysis can be subdivided into heterogeneous metal catalysts and homogeneous metal catalysts.

Catalytic supports include titanium dioxide, calcium carbonate, tungstate-zirconia, silica, alumina, silica-alumina, zirconia, cerium oxide, carbon, and USY zeolite having one or more of Ti, Zr and/or Hf substituting aluminum atoms constituting the zeolite framework. In this regard, any support known in the art may be used without limitation

In one embodiment, the hydrogenation catalyst comprises one or more active metal components selected from Pt, Pd, Re Ni, Ru, Rh, Ir, Cu, Fe, an alloy of a platinum group catalyst, and a Raney-type porous catalyst.

Preferred examples of hydrogenation catalysts, but not limited to, are Ni/C, Pd/C, Ru/C, Ni/SiO₂, and Pd/SiO₂.

The acidic catalyst can be, for example, any suitable liquid acid including inorganic liquid acids and organic liquid acids, or any suitable solid acid. Exemplary liquid acids include, but are not limited to, H2SO4, CH₃SO3H, triflic acid, and p-toluene sulfonic acid. Exemplary solid acids include, but are not limited to, Amberlyst® resins (e.g. Amberlyst®-15, Amberlyst®-36), Nafion® resins (e.g., Nafion NR₅₀), Aquivion® Resins (e.g., Aquivion® PW98, Aquivion® PW79S), Zeolites (e.g. ZSM-5, HBEA, HY), and silica supported H3PO4 (P—SiO2).

In one embodiment, these metals are immersed in a carrier comprising a metal oxide or mixed metal oxide. For example, exemplary catalysts include the above metals on cerium oxide, for example, nickel immersed in cerium oxide. Other examples of the catalysts used in the processes of the present invention include nickel immersed in zirconia, nickel immersed in a mixed oxide carrier comprising tungsten and zirconium, and ruthenium or nickel immersed in tungstate zirconia.

In one embodiment the catalyst comprises one acidic catalyst selected from the group consisting of organic liquid acids, inorganic liquid acids, solid Bronsted acids, and combinations thereof. In a further embodiment, the acidic catalyst is selected from acidic resins, fluorinated resins, zeolites, phosphoric acid, phosphorous silica, orthophosphoric acid, HCl, H₂SO₄, methanesulfonic acid, p-toluene sulfonic acid, and combinations thereof.

In a further embodiment, the hydrogenation catalyst comprises a metal catalyst selected from the group consisting of Ir, Ni, Pd, Pt, Ru, Mb, Zn, Ti, V, Cr, Mn, Fe, Co, and combinations thereof. In another embodiment, such hydrogenation catalyst is supported on a support material selected from the group consisting of activated carbon, porous carbon, silica, polymeric hybrid material, and weakly acidic material, mixture of Ni/NiO, Ni/C, Pd/C, Ru/C, Ni/SiO₂, Pd/SiO₂, Ni on zeolite supports, Ni/ZSM5, WR Grace-Raney Ni 2800, WR Grace-Raney Ni 3200, and a mixture of Ni/NiO with Al₂O₃, ZrO₂, Kieselguhr, Cr₂O₃ in different ratios.

In one embodiment, the hydrodeoxygenation catalyst comprises a metal selected from a hydrogenation metal or a base metal, and an acid site selected from a Lewis acid or a Bronsted acid, or a mixture thereof.

In a further embodiment, the hydrodeoxygenation catalyst is:
(i) a bifunctional metal-acid catalyst co-existing with the metal and acid sites selected from the group consisting of Ni/ZSM5, Ni/zeolite, Ni/silica, Ni/Al₂O₃, Raney Ni 2800, Raney Ni 3200, Pd/ZSM5, Pd/zeolite, Pd/silica, Pd/Al₂O₃, a mixture of Ni/NiO with Al₂O₃, ZrO₂, Kieselguhr, Cr₂O₃ with different ratios or a physically mixed catalyst consisting of a hydrogenation catalyst and an acid catalyst, or a supported metal-metal oxide catalyst of a general formula M¹MO wherein M¹═Ir, Ru, Ni, Co, Pd, or Rh and M=Re, Mo, W, Nb, Mn, V, Ce, Cr, Zn, Co, Y, or Al. or
(ii) a physically-mixed catalyst comprising a hydrogenation catalyst and an acid catalyst, or
(iii) a supported metal-metal oxide catalyst of a general formula M¹MO,
wherein M¹═Ir, Ru, Ni, Co, Pd, or Rh; and
M=Re, Mo, W, Nb, Mn, V, Ce, Cr, Zn, Co, Y, or Al.

### Cosmetic Compositions

By “cosmetic composition” of the invention is meant a cosmetic formulation or a cosmetic formulation precursor that comprises at least one compound of the present invention or at least one compound of the present invention made by the processes of the present invention.

By “cosmetic formulation” is meant a formulation that comprises at least one compound of the present invention, including the unsaturated compounds, the saturated compounds, and the branched compounds. The cosmetic formulation can be used for skin care, as decorative cosmetics, or personal care cosmetics.

By “cosmetic formulation precursor” is meant a formulation that may undergo additional modification, for example, by addition of one or more ingredients, or physical or chemical treatment to render it into a “cosmetic formulation” for use in skin care, decorative cosmetics, or personal care cosmetics.

The cosmetic composition of the present invention is used for skin care, skin protection in products of decorative cosmetics, or personal care cosmetics. For example, the cosmetic composition of the invention is preferably used as, or in the preparation of, emollients, lotions, water-in-oil emulsions, oil-in-water emulsions, creams, gels or flexible solids of the present invention. The cosmetic composition of the present invention is preferably used for skin care, skin protection of sensitive skin, or personal care.

Examples for personal care products comprising the cosmetic composition of the present invention include skin care cosmetics, moisturizing preparations, ointments, oils, cleansers, make-up removers, night and day treatments, skin reparatives, facial sun defense, sunscreens, blush, liquid blush, transparent blush, perfecting primer, and liquid soaps. It also includes other cosmetic product which should repair, moisturize, smooth, condition, protect, clean and rejuvenate the skin, prevent the loss of moisture, and reverse damages of the skin. Examples for products of decorative cosmetic are color foundations, make-ups, concealing sticks, rouge, tanning creams, eye shadow creams or every other cosmetic product which should color or lighten the skin.

The cosmetic composition may also comprise an effective amount of one or more additives such as pigment, fragrance, emulsifier, wetting agent, thickener, emollient, rheology modifier, viscosity modifier, gelling agent, antiperspirant agent, deodorant active, fatty acid salt, film former, anti-oxidant, humectant, opacifier, monohydric alcohol, polyhydric alcohol, fatty alcohol, preservative, pH modifier, a moisturizer, skin conditioner, stabilizing agent, proteins, skin lightening or darkening agents, topical exfoliants, antioxidants, retinoids, refractive index enhancer, photo-stability enhancer, SPF improver, UV blocker, antibiotic agents, antiseptic agents, antimicrobial agents, antifungal agents, corticosteroid agents, anti-acne agents, and water.

The cosmetic composition may be used as or in the preparation of, emollients, lotions, water-in-oil emulsions, oil-in-water emulsions, creams, gels, flexible solids, skin care cosmetics, moisturizing preparations, ointments, oils, cleansers, make-up removers, night and day treatments, skin reparatives, facial sun defense, sunscreens, blush, liquid blush, transparent blush, perfecting primer, liquid soaps, color foundations, make-ups, concealing sticks, rouge, tanning creams, eye shadow creams, shampoo, body wash, facial cleanser, face mask, bubble bath, intimate wash, bath oil, cleansing milk, micellar water, cleansing wipes, hair mask, perfume, shaving soap, shaving foam, cleansing foam, day cream, anti-ageing cream, body milk, body lotion, body mousse, face serum, eye cream, sunscreen lotion, sun cream, face cream, after-shave lotion, pre-shaving cream, depilatory cream, skin-whitening gel, self-tanning cream, anti-acne gel, mascara, foundation, primer, concealer, blush, bronzer, blemish balm cream, eyeliner, night cream, eye brow gel, highlighter, lip stain, hand sanitizer, hair oil, nail varnish remover, conditioner, hair styling gel, hair styling cream, anti-frizz serum, scalp treatment, hair colorant, split end fluid, deodorant, antiperspirant, baby cream, insect repellent, hand cream, sunscreen gel, foot cream, exfoliator, body scrub, cellulite treatment, bar soap, cuticle cream, lip balm, hair treatment, eye shadow, bath additive, body mist, eau de toilette, mouthwash, toothpaste, lubricating gel, moisturizer, serum, toner, aqua sorbet, cream gel, styling mousse, dry shampoo, lip stick, lip gloss, hydro-alcoholic gel, body oil, shower milk, illuminator, lip crayon, hair spray, combing cream, sunblock, cosmetic product that colors the skin, cosmetic product that lightens the skin, cosmetic products that repairs the skin, cosmetic product that moisturizes the skin, cosmetic product that smooth the skin, cosmetic product that conditions the skin, cosmetic products that protects the skin, cosmetic product that cleans the skin, cosmetic product that rejuvenates the skin, cosmetic product that prevents the loss of moisture, and cosmetic product that reverses damages of the skin.

For brevity, cosmetic, dermatological or pharmaceutical composition is referred to simply as “cosmetic composition” herein.

In at least one embodiment, the composition is for treating keratinous material, preferably for treating hair and/or skin. In at least one embodiment, the use of the present compound is as a thickening agent or rheology modifier in a cosmetic, dermatological or pharmaceutical composition.

In at least one embodiment, the composition comprises a cosmetically acceptable component. Suitable cosmetically acceptable components are the cosmetically acceptable component is selected from the group consisting of surfactants, auxiliaries, hair conditioning agents, hairstyling polymers, and combinations thereof. Surfactants, auxiliaries, hair conditioning agents and hairstyling polymers are disclosed in the second aspect—such cosmetically acceptable components are compatible and combinable with the first aspect.

In one embodiment, this invention relates to cosmetic compositions comprising branched-chain aliphatic hydrocarbon emollients of the present invention exhibiting ease of formulation of both oil-in-water and water-in-oil emulsions for personal care applications in which the cosmetic compositions comprising hydrocarbon emollients, water, emulsifier, and additives is formed as a stable cosmetic emulsions at cold conditions without requiring high mixing energy.

In another embodiment, this invention relates to cosmetic compositions comprising branched-chain aliphatic hydrocarbon emollients of the present invention showing excellent pigment wetting properties in which one or more branched-chain aliphatic hydrocarbon emollients exhibit excellent wettability of commercial pigments such as titanium oxide and iron oxide.

In yet another embodiment, this invention relates to cosmetic compositions comprising a series of branched chain hydrocarbon emollients allowing for the formulation of aesthetically appealing in terms of characteristics such as glossiness, firmness, stringiness, stickiness, spreadability, sliminess, and absorbency in oil-in-water and water-in-oil emulsions.

In one embodiment, this invention relates to cosmetic compositions comprising emollients of the present invention, with moisture barrier potential for acting as moisturizing agents.

In one embodiment, this invention relates to cosmetic compositions comprising the branched chain saturated furan hydrocarbons, e.g., RKsat-n, as potential solvents for sunscreen formulation, i.e., for solubility of active of sunscreen active ingredients such as avobenzone.

In one embodiment, this invention relates to branched-chain aliphatic hydrocarbon emollients exhibiting ease of formulation of both oil-in-water and water-in-oil emulsions for personal care applications. In another embodiment, the branched-chain aliphatic hydrocarbon emollients of the present invention show excellent pigment wetting properties. In yet another embodiment, this invention relates to a series of branched chain hydrocarbon emollients allowing for the formulation of aesthetically appealing oil-in-water and water-in-oil emulsions. In one embodiment, this invention relates to emollients with moisture barrier potential based on the molecular weight and lack of polarity. In another embodiment, this invention relates to branched chain saturated mono-aromatic hydrocarbons as solvents for cosmetic use active ingredients.

In one embodiment of the invention, this invention relates to cosmetic compositions comprising at least one of RK-unsat-n, RKsat-n, and RKBA-n, wherein n ranges from 10-40 carbons. Stated differently, the number of carbons is any one number selected from the following numbers:
11, 12, 13, 14, 15, 16, 17, 18, 19, 20, 21, 22, 23, 24, 25, 26, 27, 28, 29, 30, 31 ,32 33, 34 3,5 36, 37, 38, 39, and 40.

### EXPERIMENTAL

The compositions and the process of making of the aforementioned compounds are exemplified below. As described below, cosmetic formulation precursors, cosmetic formulations, and cosmetic compositions were prepared from the compounds of the present invention. The efficacy, key properties, bio-based carbon content, patch testing, pH, stability, and compatibility of emollients were evaluated. The application of the compounds has been demonstrated in the formulation of cosmetic products to evaluate formulation performance and their aesthetic profile.

### I. Condensation Reactions—Preparation of Unsaturated Compounds

Example 1: The C17 Unsaturated Product—Synthesis of (5,5′-(heptane-1,1-diyl)bis(2-methylfuran)) or RKunsat-17, by Catalytic Condensation of Heptanal and 2-Methylfuran

RKunsat-17 molecular formula=C₁₇H₂₄O₂

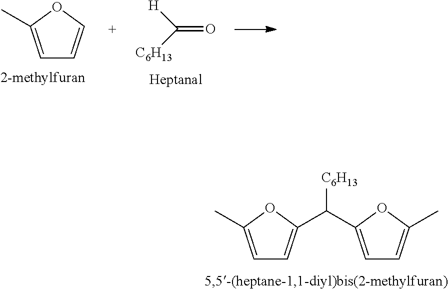

RKunsat-17 was prepared by catalytic condensation between (i) commercially procured heptanal that was biomass-derived and/or rapeseed-derived, and (ii) 2-methylfuran (2MF), in the temperature range of 65° C. to 80° C. As an acid catalyst for the condensation reaction, an in-house prepared P—SiO₂ catalyst (P=phosphorous) with 10 wt.% phosphoric acid loading (3.2% P) or a commercially procured fluorinated-resin catalyst, Aquivion® PW98, was used. The P—SiO₂ catalyst was prepared by impregnation of an aqueous solution of phosphoric acid on mesoporous silica followed by drying at 100° C. for 6 hours and calcination for 8 hours in the temperature range of 250° C. to 500° C. A molar ratio of 3:1 was used between the 2-methylfuran and heptanal to shift the reaction equilibrium towards completion and to ensure that the product did not contain significant amount of residual heptanal, which also avoids the complexity of separating the product from the final product mixture. The reaction temperature was sequentially increased—60° C. for first 30 min; 70° C. for next 30 min; and 80° C. for the rest of the reaction time—to reduce the polymerization of 2MF on the catalyst's surface in the beginning phase of the reaction. A Dean-Stark apparatus was used for the separation of water-co-product during the reaction.

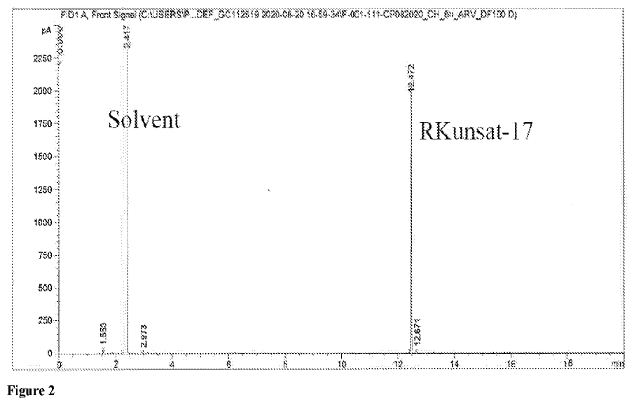

Table 1 summarizes representative results using both catalysts. It shows that the P—SiO₂ catalyst is more effective to achieve optimal yield of RKunsat-17 with a higher selectivity. After the reaction, the product was vacuum-distilled in the temperature range of 50° C. to 70° C. to remove the remaining 2MF and the water co-product. The purity of RKunsat-17 was over 99% on the basis of GC analysis (FIG. 2). The color of the product was light greenish-orange.

TABLE 1
Production of RKunsat-17 on Different Scales
Under Various Reaction Conditions

| | Reac- | | | Yield of | |
|---|---|---|---|---|---|
| | tion | | Catalyst and | RKunsat- | Conversion |
| No. | Scale | | Reaction Conditions | 17 | of Heptanal |
| 1.1 | 200 | g | P—SiO₂ 60° C. for 0.5 h; | >99% | >99% |
| | | | 70° C. for 0.5 h; | | |
| | | | and 80° C. for 4 h | | |
| 1.2 | 130 | g | P—SiO₂ 60° C. for 0.5 h; | >99% | >99% |
| | | | 70° C. for 0.5 h; | | |
| | | | and 80° C. for 4 h | | |
| 1.3 | 130 | g | Aquivion ® PW98 | 98% | 98% |
| | | | 60° C. for 0.5 h; | | |
| | | | 70° C. for 0.5 h; | | |
| | | | and 80° C. for 5 h | | |
| 1.4 | 130 | g | Aquivion ® PW98 | 98% | >98% |
| | | | 60° C. for 2 h; | | |
| | | | and 80° C. for 7 h | | |
| 1.5 | 70 | g | Aquivion ® PW98 | 99% | >99% |
| | | | 60° C. for 2 h; | | |
| | | | and 80° C. for 5 h | | |
| 1.6 | 10 | g | Aquivion ®PW98 | 97% | >97 |
| | | | 80° C. for 4 h | | |
| 1.7 | 10 | g | P—SiO₂; 65° C. for 5.5 h | 80% | 81% |

Example 2: The C15 Unsaturated Product—Synthesis of 5,5(Furan-2-ylmethylene)bis(2-methylfuran) (RKunsat-15) by Catalytic Condensation of Furfural and 2-Methylfuran

Molecular Formula of RKunsat-15=C₁₅H₁₄O₃

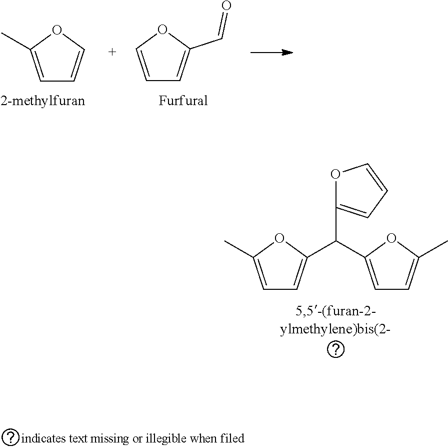

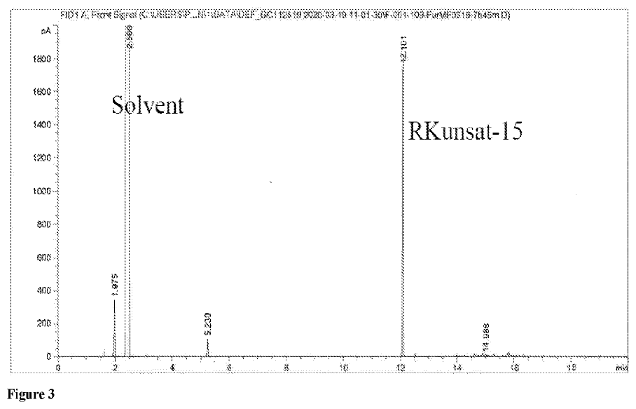

RKunsat-15 was prepared by catalytic condensation between commercially procured, biomass-derived furfural and 2-methylfuran (2MF) in the temperature range of 65° C. to 80° C. The experimental and product purification methodologies were similar to those of the aforementioned RKunsat-17 product. The staged heating enabled improved yield and product selectivity. In addition, fluorinated Aquivion® PW98 and P—SiO₂ catalysts were used in the present reaction, which were found to be more effective, selective, and stable. Table 2 summarizes representative results. It shows that P—SiO₂ catalyst was more effective to achieve optimal yields and selectivity of RKunsat-15. The purity of RKunsat-15 after distillation was over 99% on the basis of GC analysis (FIG. 3). The color of the product was reddish.

TABLE 2
Production of RKunsat-15 on Different Reaction
Scales Under Various Reaction Conditions

| | Reac- | | | Yield of | |
|---|---|---|---|---|---|
| | tion | | Catalyst and | RKunsat- | Conversion |
| No. | Scale | | Reaction Conditions | 15 | of Furfural |
| 2.1 | 5 | g | P—SiO₂ | 66% | 67% |
| | | | 65° C. for 4 h | | |
| 2.2 | 40 | g | Aquivion ®PW98 | 90% | 92% |
| | | | 65° C. for 8.5 h | | |
| 2.3 | 200 | g | Aquivion ®PW98 | 90% | 93% |
| | | | 65° C. for 8 h | | |

Example 3: The C22 Product—Synthesis of 5,5′-(Dodecane-1,1-diyl)bis(2-methylfuran) (RKunsat-22) by Catalytic Condensation of Dodecanal and 2-Methylfuran

Molecular Formula of RKunsat-22=C₂₂H₃₄O₂

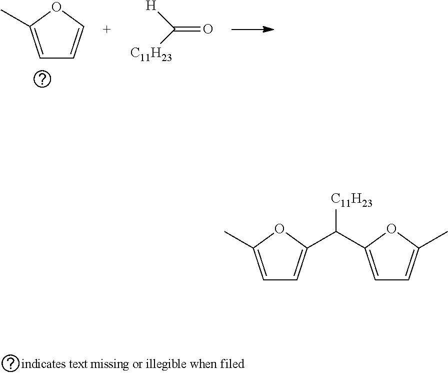

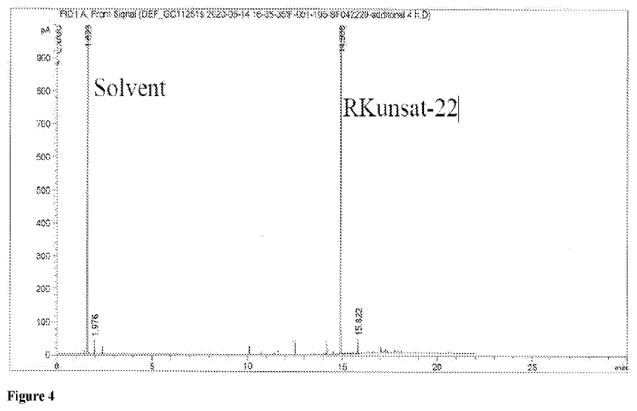

RKunsat-22 was prepared by catalytic condensation between commercially procured, biomass-derived 2MF and a fatty acid-derived and/or rapeseed-pyrolyzed dodecanal in the temperature range of 65° C. to 80° C. The experimental and product purification methodologies were similar to those of the aforementioned RKunsat-17 product. The staged heating enabled better yield and product selectivity. Table 3 summarizes representative results. It shows that P—SiO₂ catalyst is more effective in achieving optimal yields of RKunsat-22 at higher selectivity. The purity of RKunsat-22 after distillation was greater than 99% on the basis of GC analysis (FIG. 4). The color of the product was light orange.

The synthesis of RKunsat-22 was scaled to 33 kg through the condensation of 2-methylfuran and lauraldehyde in a 72 L glass reactor. The reactor was equipped with a mechanical stirrer, liquid circulatory jacket for controlling desired reaction temperature, an outlet for sampling during the reaction and product recovery, pumps for adding reactants into the reactor, and a Dean-Stark set up for capturing water co-product during the reaction. Lauraldehyde in the amount of 16.2 kg was reacted with 24.3 kg of 2-methylfuran in the presence of 1.2 kg in-house-produced P—SiO₂ catalyst. First, the reaction was initiated at 50° C. and was held for about 30 min. The, the temperature was ramped up to 60° C. with a hold time of ½ h. The initial reaction at lower temperature facilitated the production of the condensation product at a faster rate without an equilibrium limitation by the water co-product. About 50% reaction was completed within 1 h. The lower reaction temperature, initially, also helped eliminate self-condensation of 2-methylfuran into the polymeric product. Subsequently, the reaction temperature was raised gradually to 70° C. and then to 82° C. to shift the reaction equilibrium in the forward direction. Water formation was observed, and the water was separated and removed from the hydrophilic catalyst. Finally, RKunsat-22 with 95% purity was obtained. The catalyst was separated through a 0.5-micron filter. Subsequently, the excess 2-methylfuran and the water co-product were stripped off.

TABLE 3
Production of RKunsat-22 on Different Reaction
Scales Under Various Reaction Conditions

| | | | | | Conver- |
|---|---|---|---|---|---|
| | | | | Yield of | sion of |
| | Reaction | | Catalyst and | RKunsat- | laur- |
| No. | Scale | | Reaction Conditions | 22 | aldehyde |
| 3.1 | 1,300 | g | Aquivion ® PW98 | 92% | >95% |
| | | | 65° C. for 10 h | | |
| 3.2 | 175 | g | P—SiO₂ 60° C. for 0.5 h; | >99% | >99% |
| | | | 70° C. for 0.5 h; | | |
| | | | and 80° C. for 5.5 h | | |
| 3.3 | 175 | g | Aquivion ® PW98 | 94% | 95% |
| | | | 60° C. for 0.5 h; | | |
| | | | 70° C. for 0.5 h; | | |
| | | | and 80° C. for 8.5 h | | |
| 3.4 | 130 | g | Aquivion ® PW98 | 82% | 83% |
| | | | 70° C. for 8 h | | |
| 3.5 | 130 | g | Aquivion ®PW98 | 98% | >98% |
| | | | 60° C. for 2 h, | | |
| | | | and 80° C. for 7 h | | |
| 3.6 | 140 | g | Aquivion ®PW98 | 70% | 71% |
| | | | 65° C. for 5 h | | |
| 3.7 | 33,000 | g | P—SiO₂ 50° C. for 0.5 h; | 95% | >99% |
| | | | 60° C. for 0.5 h; | | |
| | | | 70° C. for 1 h | | |
| | | | and 82° C. for 5.5 h | | |

### II. Hydrogenation Reactions—Preparation of Saturated Compounds

Example 4: The C17 Product—Synthesis of 5,5′-(Heptane-1,1-diyl)bis(2-methyltetrahydrofuran) (RKsat-17) by Catalytic Hydrogenation of RKunsat-17

Molecular Formula of RKsat-17=C₁₇H₃₂O₂

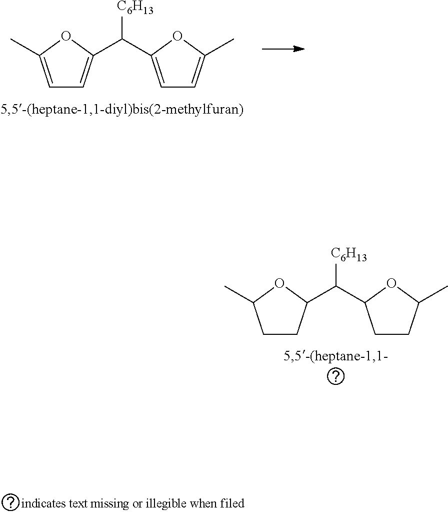

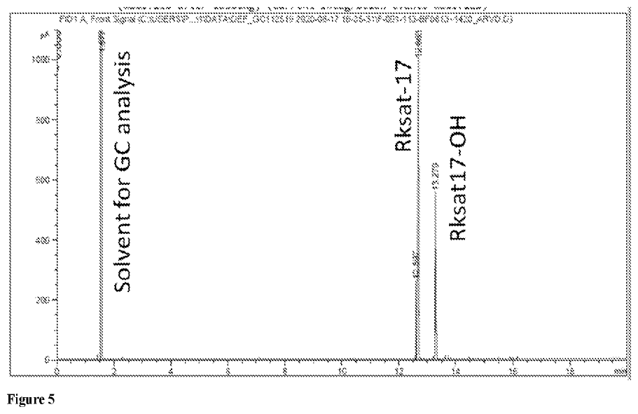

RKsat-17 product was prepared by catalytic hydrogenation of RKunsat-17 in a solvent, using a Parr reactor. The reactor was first purged with N₂ before adding H₂ gas. Heptane and cyclohexane were used as solvents and the hydrogenation performance was similar in both solvents. Commercially procured palladium on activated-carbon with 5% and 10% loadings (Pd/C with 5 wt. % Pd loading; Pd/C with 10% Pd loading) were tested and the catalyst with 5% Pd loading was found to be more effective to produce RKsat-17 with higher selectivity. Table 4 shows the results. After the reaction, the solvent was removed by distillation. Any residual solvent and small amounts of low carbon number products were removed by vacuum distillation in the temperature range of 40-60° C. The main product was RKsat-17 with a small amount (10-25%) of a hydroxyl product of RKsat-17 (referred hereto as RKsat-17-OH), which was formed upon ring-opening of one of the two cyclo-ether rings of RKsat-17. GC (FIG. 5) and GC-MS data indicate that the purity of the product containing the two components was 99%. The product was colorless and odorless.

TABLE 4
Production of RKsat-17 from Hydrogenation of
RKunsat-17 Under Various Reaction Conditions

| | | | Ratio of | |
|---|---|---|---|---|
| | Reaction | | RKsat-17 | |
| | Scale | Catalyst | to RKsat-17- | Yield |
| No. | Per Batch | Reaction Conditions | OH | (Purity) |
| 4.1 | 70 g | Pd/C (5 wt. % Pd) | 76:24 | >99% |
| | | 60° C., 2 h, 40 bar H₂ | | |
| | | substrate to solvent | | |
| | | volume ratio = 0.14 | | |
| 4.2 | 60 g | Used Pd/C (5 wt. % Pd) | 81:19 | >99% |
| | | 60° C., 2 h, 40 bar H₂ | | |
| | | substrate to solvent | | |
| | | volume ratio = 0.12 | | |
| 4.3 | 48 g | Pd/C (10 wt. % Pd) | 63:37 | >99% |
| | | 60° C., 2 h, 40 bar H₂ | | |
| | | substrate to solvent | | |
| | | volume ratio = 0.1 | | |
| 4.4 | 30 g | Pd/C (5 wt. % Pd) | 69:31 | >99% |
| | | 60° C., 1 h, 40 bar H₂ | | |
| | | substrate to solvent | | |
| | | volume ratio = 0.1 | | |

Example 5: The C22 Product—Synthesis of 5,5′-(Dodecane-1,1-diyl)bis(2-methyltetrahydrofuran) (RKsat-22) by Catalytic Hydrogenation of RKunsat-22

Molecular Formula of RKsat-22=C₂₂H₄₂O₂

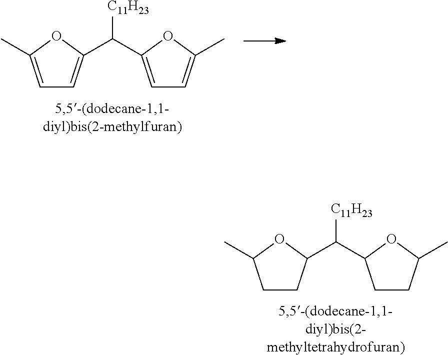

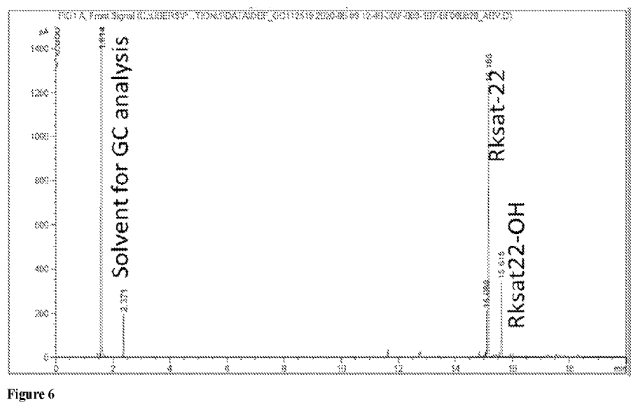

RKsat-22 product was prepared by catalytic hydrogenation of RKunsat-22 in cyclohexane using a Parr reactor. The reactor was first purged with N₂ before adding H₂ gas. Commercially procured palladium on activated-carbon with 5% and 10% loadings (Pd/C with 5 wt. % Pd loading; Pd/C with 10 wt. % Pd loading) were tested, and the catalyst with 5 wt. % Pd loading was found to be more effective to produce RKsat-22 at higher selectivity. Table 5 shows the results. The purification of product was similar to RKsat-17 in Example 4. The main product was RKsat-22 with a small amount (15-25%) of a hydroxyl product of RKsat-22 (referred hereto as RKsat22-OH), which was formed upon ring-opening of one the two cyclo-ether rings of RKsat-22. GC (FIG. 6) and GC-MS data indicated that the purity of the product containing the two components varied in the range of 95-99%. The product was colorless and odorless.

TABLE 5
Production of RKsat-22 from Hydrogenation of
RKunsat-22 Under Various Reaction Conditions

| | | | Ratio of | |
|---|---|---|---|---|
| | Reaction | | RKsat-22 | |
| | Scale | Catalyst | to RKsat-22- | Yield |
| No. | Per Batch | Reaction Conditions | OH | (Purity) |
| 5.1 | 340 g | Pd/C (10 wt. % Pd) | 75:25 | >99% |
| | | 80° C., 16 h, 37 bar H₂ | | |
| | | substrate to solvent | | |
| | | volume ratio = 0.14 | | |
| 5.2 | 60 g | Pd/C (5 wt. % Pd) | 76:22 | >99% |
| | | 60° C., 1 h, 40 bar H₂ | | |
| | | substrate to solvent | | |
| | | volume ratio = 0.12 | | |
| 5.3 | 30 g | Pd/C (5 wt. % Pd) | 85:15 | 98% |
| | | 60° C., 1 h, 40 bar H₂ | | |
| | | substrate to solvent | | |
| | | volume ratio = 0.1 | | |
| 5.4 | 30 g | Pd/C (5 wt. % Pd) | 81:19 | 99% |
| | | 60° C., 2 h, 40 bar H₂ | | |
| | | substrate to solvent | | |
| | | volume ratio = 0.1 | | |
| 5.5 | 30 g | Pd/C (5 wt. % Pd) | 80:20 | 99% |
| | | 60° C., 3 h, 40 bar H₂ | | |
| | | substrate to solvent | | |
| | | volume ratio = 0.1 | | |
| 5.6 | 70 g | Pd/C (5 wt. % Pd) | 79:21 | 95% |
| | | 60° C., 1 h, 40 bar H₂ | | |
| | | substrate to solvent | | |
| | | volume ratio = 0.14 | | |

Example 6: The C15 Product—Synthesis of 5,5″((tetrahydrofuran-2-yl)methylenebis(2-methylene-tetrahydrofuran)(RKsat-15) by Catalytic Hydrogenation of RKunsat-15

Molecular Formula of RKsat-15: C₁₅H₂₆O₃

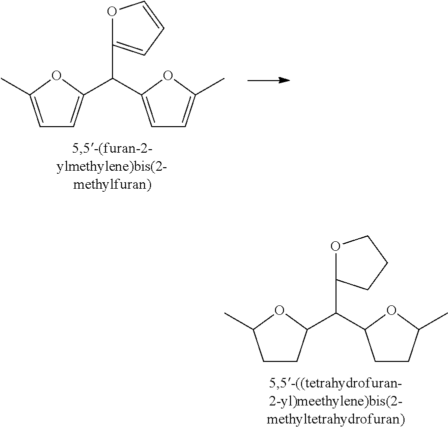

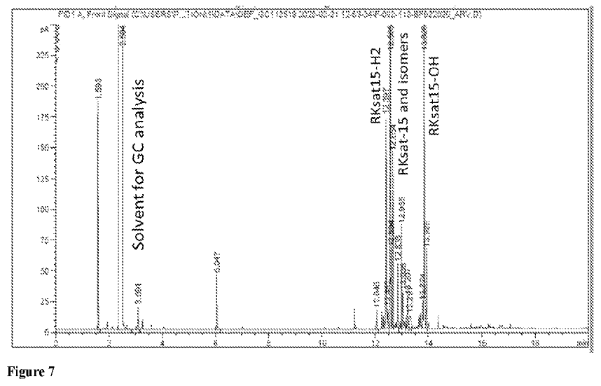

RKsat-15 product was prepared by catalytic hydrogenation of RKunsat-15 in cyclohexane using a Parr reactor. The reactor was first purged with N₂ before adding H₂ gas. Commercially procured palladium on activated-carbon with 10% Pd loadings (Pd/C with 10% Pd loading) was used. Table 6 summarizes the results. The purification of the product was similar to that of RKsat-17. GC and GC-MS indicated that the main product was RKsat-15 with a small amount of hydroxyl products of RKsat-15 with or without olefinic bond (referred hereto as RKsat-15-OH), which are formed upon ring opening of one or more cyclo-ether rings of RKsat-15. GC profile of the product is shown in FIG. 7. The product was light orange.

TABLE 6
Production of RKsat-15 from Hydrogenation of
RKunsat-15 Under Various Reaction Conditions

| | Reaction | | | | |
|---|---|---|---|---|---|
| | Scale | Catalyst | RKsat- | RKsat- | RKsat- |
| No. | Per Batch | Reaction Conditions | 15 | 15-OH | 15-H2* |
| 6.1 | 20 g | Pd/C (10 wt. % Pd)) | 45% | 31% | 20% |
| | | 80° C., 40 min, 40 H₂ | | | |
| | | substrate to solvent | | | |
| | | volume ratio = 0.08 | | | |
| 6.2 | 20 g | Pd/C (5 wt. % Pd)) | 45% | 23% | 23% |
| | | 60° C., 1.5 h, 40 bar H₂ | | | |
| | | substrate to solvent | | | |
| | | volume ratio = 0.08 | | | |

*RKsat-15-H2 contains two tetrahydrofuran rings and one furan ring.

### III. Hydrodeoxygenation Reactions—Preparation of Branched Hydrocarbon Compounds

Example 7: C17 Product—Synthesis of 6-Pentyldodecane (RKBA-17) by Catalytic Hydrodeoxygenation of RKunsat-17

Molecular Formula of RKBA-17: C₁₇H₃₆

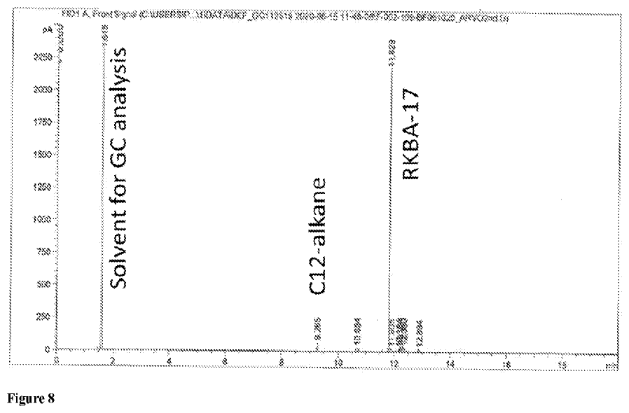

RKBA-17 product was prepared by catalytic hydrodeoxygenation of RKunsat-17 in cyclohexane as a solvent using a Parr reactor. The reactor was first purged with N₂ before adding H₂ gas. Three different catalysts containing metal and acid sites (physical mixture of commercially procured Pd/C with 10 wt. % Pd loading and hafnium (IV) triflate; in-house produced Ni/ZSM-5 (20 wt. % Ni); and in-house produced Pd/ZSM-5 (5 wt. % Pd)) were tested. Ni/ZSM5 and Pd/ZSM5 catalysts were pre-pared by slow impregnation of Ni-nitrate and Pd-nitrate aqueous solutions over Zeolyst CBV 2314 ZSM-5 support, respectively, followed by drying and calcination in a furnace. An organic reagent, ethylene glycol, was used for uniform dispersion of the metal sides on the ZSM-5 support. After the reaction, the solvent was removed by distillation. Any residual solvent and low carbon-number alkanes were removed by vacuum distillation in the temperature range of 40-60° C. The main product was RKBA-17 along with a small amount of its alkene isomer (referred here to as RKBA-17-isomer). The product was qualitatively and quantitatively analyzed by GC and GCMS. The results are summarized in Table 7. GC data indicate the purity of the product containing the two components was 99% by the Pd/C-Hf(OTf)₄ catalyst. The Ni/ZSM5 and Pd/ZSM5 catalysts produced small amount of a linear C₁₂-alkane because of a C—C cleavage during the hydrodeoxygenation reaction. The C₁₂-alkane was flashed off under vacuum distillation to obtain nearly 100% pure RKBA-17 (FIG. 8). The product was colorless.

Hydrodeoxygenation of RKunsat-17 was also conducted using commercially procured Ni catalyst, JM-Ni62/15P (JM=Johnson Matthey) with or without using a solvent. The catalyst was pre-activated at 160° C. under 5 bar H₂. Several reactions were conducted. A few representative reactions, their experimental conditions, and the results are shown in Table 7 (No. 7.5 to 7.10).

TABLE 7
Production of RKBA-17 from Hydrodeoxygenation
of RKunsat-17 Under Various Reaction Conditions

| | Reaction | | | | |
|---|---|---|---|---|---|
| | Scale | Catalyst | RKBA- | RKBA-17- | C12- |
| No. | Per Batch | Reaction Conditions | 17 | iso-mer | al-kane |
| 7.1 | 50 g | Pd/C (10 wt. % Pd) | 92% | 7.2% | 0 |
| | | (5.8 g), Hf(OTf)₄ (2.6 g) | | | |
| | | 200° C., 12 h, 40 bar H₂ | | | |
| | | substrate to solvent volume | | | |
| | | ratio = 0.125. | | | |
| 7.2 | 30 g | Ni/ZSM5 (4 g) | 91% | 0 | 9% |
| | | 225° C., 15 h, 40 bar H₂ | | | |
| | | substrate to solvent volume | | | |
| | | ratio = 0.1 | | | |
| 7.3^{a} | 30 g | Pd/ZSM5 (4 g) | 76% | 7.7% | 10.8% |
| | | 200° C., 12 h, 40 bar H₂ | | | |
| | | substrate to solvent volume | | | |
| | | ratio = 0.15 | | | |
| 7.4^{b} | 30 g | Pd/ZSM5 (4 g) | 45% | 13% | 6.6% |
| | | 200° C., 12 h, 24 bar H₂ | | | |
| | | substrate to solvent volume | | | |
| | | ratio = 0.15 | | | |
| 7.5^{c} | 150 g | JM-Ni62/15P (5 g) | 88% | 2.3% | 6% |
| | | 240° C. for 15 h, 40 bar H₂ | | | |
| | | substrate to solvent (hexane) | | | |
| | | volume ratio = 0.27 | | | |
| 7.6^{d} | 150 g | JM-Ni62/15P (5 g) | 86% | 4% | 5.6% |
| | | 240° C. for 12 h, 40 bar H₂ | | | |
| | | substrate to solvent (hexane) | | | |
| | | volume ratio = 0.27 | | | |
| 7.7^{e} | 150 g | JM-Ni62/15P (5 g; 1-time | 81% | 3.9% | 4.6% |
| | | used catalyst) | | | |
| | | 240° C. for 15 h, 40 bar H² | | | |
| | | substrate to solvent (hexane) | | | |
| | | volume ratio = 0.27 | | | |
| 7.8^{f} | 800 g | JM-Ni62/15P (20 g) | 86% | 2% | 6% |
| | | 240° C. for 12 h, 45 bar H₂. | | | |
| | | No solvent was used. | | | |

^{a}5.1% RKsat-17 was present
^{b}29% RKsat-17 was present
^{c}3% C₁₄-C₁₆ alkanes were present
^{d}3.5% C₁₄-C₁₆ alkanes were present
^{e}balance compounds are C₁₄-C₁₆ alkanes and C₁₇ alkene
^{f}balance compounds are C₇-C₁₆ alkanes

Example 8: C22 Product—Synthesis of 6-pentylheptadecane (RKBA-22) by Catalytic Hydrodeoxygenation of RKsat-22

Provide Expanded Formula of RKBA-22: C₂₂H₄₄

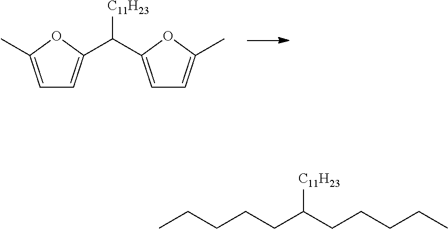

RKBA-22 product was prepared by catalytic hydrodeoxygenation of RKunsat-22 in cyclohexane as a solvent using a Parr reactor. The reactor was first purged with N₂ before adding H₂ gas. Three different catalysts containing metal and acid sites (physical mixture of commercially procured Pd/C with 10% Pd loading and hafnium (IV) triflate; in-house produced Ni/ZSM-5 (20 wt. % Ni); and in-house produced Pd/ZSM-5 (5 wt. % Pd) were tested. After the reaction, solvent was removed by distillation. Any residual solvent and low carbon-number alkanes were removed by vacuum distillation in the temperature range of 40-60° C. The main product was RKBA-22 along with a small amount of its alkene isomer (referred here to as RKBA-22-isomer).

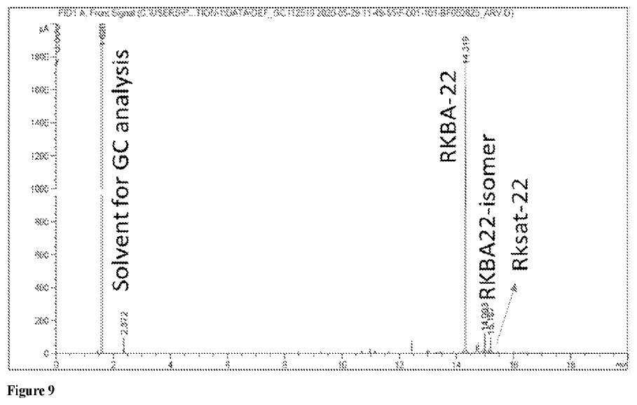

The challenge for the Pd/C catalyst is that it requires a Lewis acid, Hf(triflate)₄, which is expensive and has a supply constraint. An in-house synthesized Ni/ZSMS was used as a catalyst for the hydrodeoxygenation, which produced RKBA-17 with high yield and complete conversation of the starting material, but it is not a commercial product. Ni/ZSMS was prepared by wet impregnation of an aqueous solution of 20 wt. % nickel nitrate on ZSMS support followed by calcination at 300° C. We also studied the catalytic efficiency of five Ni-catalysts procured from WR Grace and Johnson Matthey. These are WR Grace Ni3202, WR Grace Ni 2800, JM Ni62/15P, JM Ni55/5P and JM Cu60/8P catalysts. All catalysts were pre-activated to reduce any surface NiO on the catalyst surface. The catalyst activation was conducted in a solvent (cyclohexane or hexane) or using RKunsat-22 as a solvent without using an external solvent. The reduction was done at 160° C. and 5 bar H₂ pressure for 2 h. Upon activation, a fixed amount of RKunsat-22 was added into the reactor for hydrodeoxygenation of the substrate. The product was qualitatively and quantitatively analyzed by GC and GCMS. The results are summarized in Table 8. The GC (FIG. 9) data indicated that the purity of the product containing the two components was 82%-99% by the Pd/C-Hf(OTf)₄ catalyst. Ni/ZSM5 catalyst produced small amounts of a C₁₇ alkane (heptadecane) because of C—C cleavage during the hydrodeoxygenation reaction. The product was colorless and odorless.

Table 8 (No. 8.7 to 8.11) results indicate that the JM Ni62/15P catalyst is the most effective in terms of conversion of the substrate and the yield of RKBA-22. Only 8% heptadecane was formed via C—C cleavage, which can also be used for cosmetic or lubricant formulations. JM Ni62/15P catalyst composition consists of Ni 30 wt. %, NiO 37 wt. %, ZrO₂2 wt. %, Al₂O₃3.7 wt. % and Kieselguhr 27 wt. %. Upon activation, the NiO sites become Ni and the Ni hydrogenates the furan rings of RKunsat-22. A small number of acidic sites from alumina then facilitates the ring opening chemistry of the hydrogenated furan rings to the hydroxylated intermediates. The hydroxylated intermediates then dehydrate to olefinic intermediates by the acid sites and then hydrogenate to branched alkanes (RKBA-22). A small percentage of C—C cleavage takes place, resulting in formation of heptadecane. The high surface area of the catalyst (BET Surface Area 200 m²/g) is beneficial to the HDO reaction.

Next we varied the reaction conditions using the JM Ni62/15P catalyst to determine the best conditions for scaling up the process. Several experiments were conducted on different scales using used and new JM Ni62/15P catalyst. The reactions were also conducted under neat conditions, without using a solvent. The results are shown in Table 8 (No. 8.12 to 8.15). The results indicated that water formation during the hydrodeoxygenation reaction posed a challenge to the reaction rates for the larger scale reactions. Initially the reaction was fast. As the water co-product accumulated in the reaction solution, it was likely covering the catalyst surface which inhibited the reaction. Therefore, the reaction slowed down and consequently, the C—C cleavage resulted. This effect was more pronounced as the reaction was scaled up. A reaction was also conducted using decane as a solvent, which exhibited a similar reaction productivity as cyclohexane or hexane.

For example, a reaction in a 1-gallon reactor was where 350 g RKunsat-22 was reacted with 12 g JM Ni62/15P pre-activated catalyst in cyclohexane at 240° C. under 45 bar H₂. The reaction product was collected as a function of time. It showed that after 3.5 h of reaction, H₂ consumption was very slow. Then the reaction solution was cooled down to 110° C., the solvent along with accumulated water co-product was stripped off, new solvent was fed in, and then the reaction was resumed, again at 240° C. under 45 bar H₂. Using the new solvent, the reaction initiated again and was completed. It indicated that accumulated water was a challenge to shift the reaction equilibrium towards completion. The GC chromatogram of the product showed a total of 87% RKBA-22 along with 5.6% heptadecane and 5.6% nonadecane were produced (Table 8; No. 8.16).

The hydrodeoxygenation reaction of RKunsat-22 was further scaled up using a 30-gallon reactor. The reaction was conducted in two batches to produce 18 kg of RKBA-22 (˜9 kg per batch). In the first batch, the reaction was conducted following the same methodology used for the reaction in 1 gallon reactor. The H₂ consumption results indicated that the reaction was slower after 3.5 h of reaction and until the water co-product was stripped off by the separation of solvent along with water and then with an addition of new solvent. In the second batch, the reaction was therefore conducted first for 15 h at 240° C. under 45 bar H₂ and then water along with solvent was stripped off under vacuum. New cyclohexane was added and then the reaction continued on for another 2 h at 240° C. under 45 bar of H₂. Solvent, and any light alkanes were stripped off by vacuum distillation to obtain the final RKBA-22 (Table 8; No. 8.17).

TABLE 8
Production of RKBA-22 from Hydrodeoxygenation
of RKunsat-22 Under Various Reaction Conditions

| | Reaction | | | | | |
|---|---|---|---|---|---|---|
| | Scale Per | | Catalyst and | RKBA- | RKBA-22- | C17- |
| No. | Batch | | Reaction Conditions | 22 | iso-mer | al-kane |
| 8.1 | 580 | g | Pd/C (10 wt. % Pd) (120 g), | 85% | 15% | 0 |
| | | | Hf(OTf)₄ (58 g) | | | |
| | | | 220° C., 16 h, 44 bar H₂ | | | |
| | | | substrate to solvent vol | | | |
| | | | ratio = 0.23 | | | |
| 8.2 | 50 | g | Pd/C (10 wt. % Pd) (7 g), | 98% | 2% | 0 |
| | | | Hf(OTf)₄ (3 g) | | | |
| | | | 200° C., 15 h, 40 bar H₂ | | | |
| | | | substrate to solvent volume | | | |
| | | | ratio = 0.1 | | | |
| 8.3^{a} | 60 | g | Pd/C (10 wt. % Pd) (6 g), | 32% | 50% | 0 |
| | | | Hf(OTf)₄ (2.5 g) | | | |
| | | | 200° C., 12 h, 40 bar H₂ | | | |
| | | | substrate to solvent volume | | | |
| | | | ratio = 0.11 | | | |
| 8.4^{b} | 60 | g | Pd/C (10 wt. % Pd) (7 g), | 90% | 5.6% | 0 |
| | | | Hf(OTf)₄ (3 g) | | | |
| | | | 225° C., 15 h, 40 bar H₂ | | | |
| | | | substrate to solvent volume | | | |
| | | | ratio = 0.11 | | | |
| 8.5 | 30 | g | Ni/ZSM5 (4 g) | 84.2% | 5% | 11.8% |
| | | | 225° C., 19 h, 40 bar H₂ | | | |
| | | | substrate to solvent volume | | | |
| | | | ratio = 0.1 | | | |
| 8.6 | 30 | g | Ni/ZSM5 (3.2 g) | 90% | 0 | 10% |
| | | | 225° C. for 15 h and then | | | |
| | | | 240° C. for 5 h, 40 bar H₂ | | | |
| | | | substrate to solvent volume | | | |
| | | | ratio = 0.1 | | | |
| 8.7 | 60 | g | WR-Ni2800 (5 g) | Major product was ring-hydrogenated | | |
| | | | First at 80 C. for 4 h and then | RKsat-22 and a small amount of RKBA-22 | | |
| | | | at 225° C. for 15 h, 45 bar H₂, | | | |
| | | | substrate to solvent (hexane) | | | |
| | | | volume ratio = 0.125 | | | |
| 8.8 | 100 | g | WR-Ni3200 (5 g) | Major product was ring-hydrogenated | | |
| | | | 240° C. for 15 h, 45 bar H₂, | RKsat-22 and a small amount of RKBA-22 | | |
| | | | substrate to solvent (hexane) | along with C—C cleavage alkanes | | |
| | | | volume ratio = 0.125 | | | |
| 8.9 | 60 | g | JM-Ni62/15P (5 g) | 91% | 0 | 8% |
| | | | 240° C. for 15 h, 40 bar H₂, | | | |
| | | | substrate to solvent (cyclohexane) | | | |
| | | | volume ratio = 0.11 | | | |
| 8.10^{c} | 60 | g | JM-Ni55/5P (5 g) | 60% | 4% | 6% |
| | | | 240° C. for 15 h, 40 bar H₂, | | | |
| | | | substrate to solvent (cyclohexane) | | | |
| | | | volume ratio = 0.11 | | | |
| 8.11 | 60 | g | JM-Cu60/8P (5 g) | Major products are ring hydrogenated | | |
| | | | 240° C. for 15 h, 40 bar H₂, | RKsat-22 and their ring opened intermediates. | | |
| | | | substrate to solvent (cyclohexane) | No RKBA-22 observed. | | |
| | | | volume ratio = 0.11 | | | |
| 8.12 | 100 | g | JM-Ni62/15P (5 g) | 90% | — | 8.8% |
| | | | 240° C., 15 h, 40 bar H₂, | | | |
| | | | Substrate to solvent (hexane) | | | |
| | | | volume ratio = 0.18 | | | |
| 8.13 | 200 | g | JM-Ni 62/15P (5 g) | 76% | 11% | 5%^{a} |
| | | | 240° C. for 16 h, 40 bar H₂, | | | |
| | | | Substrate to solvent (hexane) | | | |
| | | | volume ratio = 0.36 | | | |
| 8.15 | 150 | g | JM-Ni 62/15P (5 g) | 87% | 3.7% | 7% |
| | | | 230° C. for 2 h and then | | | |
| | | | 240° C. for 13 h, 40 bar H₂, | | | |
| | | | substrate to solvent (hexane) | | | |
| | | | volume ratio = 0.27 | | | |
| 8.16^{d} | 350 | g | JM-Ni 62/15P (12 g) | 87% | 0 | 5.6% |
| | | | 240° C. for 15 h and then water | | | |
| | | | stripped off and then continued | | | |
| | | | reaction at 240° C. for 10 h, | | | |
| | | | 45 bar H₂, substrate to solvent | | | |
| | | | (cyclohexane) volume ratio = 0.27 | | | |
| 8.17^{e} | 9000 | g | JM-Ni 62/15P (1300 g) | 77% | 1.5% | 12% |
| | | | 240° C. for 15 h and then water | | | |
| | | | stripped off and then continued | | | |
| | | | reaction at 240° C. for 2 h, | | | |
| | | | 45 bar H₂, substrate to solvent | | | |
| | | | (cyclohexane) volume ratio = 0.27 | | | |
| 8.18^{f} | 700 | g | JM-Ni 62/15P (20 g) | 81% | 3% | 12% |
| | | | 240°° C. for 19 h. 45 bar H₂, water | | | |
| | | | was stripped off two times during the | | | |
| | | | reaction. No solvent was used | | | |
| 8.19^{g} | 800 | g | JM-Ni 62/15P (20 g) | 78% | 3.5% | 12.8% |
| | | | 240° C. for 19 h. 45 bar H₂, water | | | |
| | | | was stripped off two times during the | | | |
| | | | reaction. No solvent was used | | | |
| 8.20^{h} | 700 | g | JM-Ni 62/15P (20 g; 1- time used) | 72% | 7% | 12% |
| | | | 240° C. for 19 h. 45 bar H₂, water | | | |
| | | | was stripped off two times during the | | | |
| | | | reaction. No solvent was used | | | |
| 8.21ⁱ | 700 | g | JM-Ni 62/15P (20 g; 2- times used) | 72% | 6% | 14% |
| | | | 240° C. for 19 h. 45 bar H₂, water | | | |
| | | | was stripped off two times during the | | | |
| | | | reaction. No solvent was used | | | |

^{a}5.9% C15-alkane and 10.9% RKsat-22 were present
^{b}4.3% RKsat-22 was present
^{c}30% RKsat-22 was present
^{d}5.6% nonadecane.
^{e}other compounds: C₁₆-C₂₀ linear alkanes = 7.4%
^{f}other compounds: 2% undecane, 1% pentadecane
^{g}other compounds: 2.7% undecane, 1.4% pentadecane, 0.7% hexadecane
^{h}other compounds: 1% nonadecane and balance other alkanes in the range of C₁₅-C₂₀
ⁱother compounds: alkanes in the range of C₁₁-C₂₀

Hydrodeoxygenation of RKunsat-22 was also performed under neat conditions without using a solvent. In this case, the JM-Ni62/15P catalyst was first activated in RKunsat-22. After the catalyst activation, hydrogen pressure in the reactor as well as the reaction temperature was elevated. More specifically, activation was accomplished at 160° C. and 5 bar H₂. HDO reaction was conducted at 240° C. and 45 bar H₂ pressure. So the temperature was increased and H₂ was added. The water co-product was vacuum-stripped twice during the reaction. Finally the resultant colorless product was distilled off to separate light alkanes to obtain the final odor-free RKBA-22 (Table 8; No. 8.18-8.19). The catalyst was re-used three times without any loss of the activity (Table 8; No. 8.19-9.21).

Example 9: C15 Product—Synthesis of 6-Butylundecane (RKBA-15) by Catalytic Hydrodeoxygenation of RKunsat-15

Molecular Formula of RKBA-15: C₁₅H₃₂

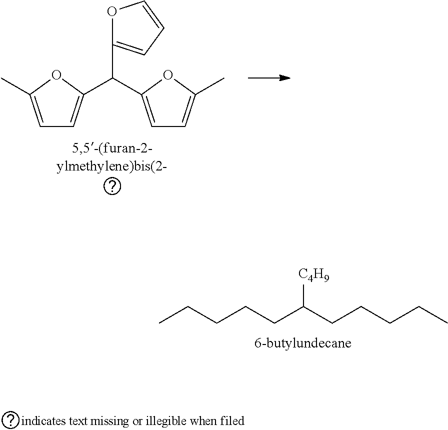

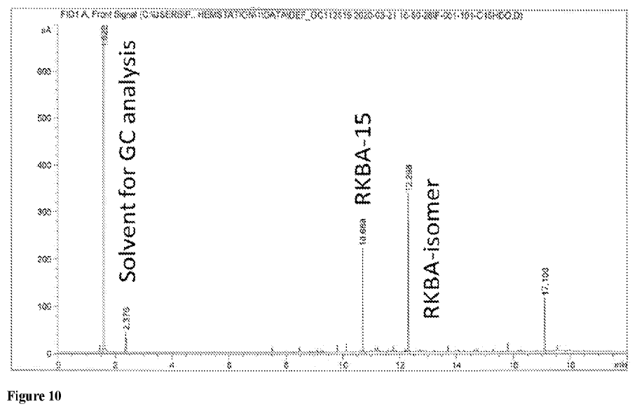

The RKBA-15 product was prepared by catalytic hydrodeoxygenation of RKunsat-15 in cyclohexane as a solvent using a Parr reactor. The reactor was first purged with N₂ before adding H₂ gas. A physical mixture of Pd/C (10 wt. % Pd loading) and hafnium (IV) triflate was used. After the reaction, the solvent was removed by distillation. Any residual solvent was removed by vacuum distillation in the temperature range of 40-60° C. The main product was RKBA-15 along with its alkene isomer or intermediate cyclic-ether ring opened product (referred here to as RKBA15-isomer). The product was qualitatively and quantitatively analyzed by GC and GCMS. The results are summarized in Table 9. A higher molecular-weight product was observed, which was likely formed via condensation of RKUnsat-15 intermediate species with a C5-intermediate, followed by reorganization. The GC (FIG. 10) data indicate the purity of the product containing two components was ˜80%. The product color light yellow.

TABLE 9
Production of RKBA-15 from Hydrodeoxygenation
of RKunsat-15 Under Various Conditions

| | Reaction | | | |
|---|---|---|---|---|
| | Scale Per | Catalyst and | RKBA- | RKBA15- |
| No. | Batch | Reaction Conditions | 15 | isomer |
| 9.1 | 30 g | Pd/C (10 wt. % Pd) (5.8 g), | 27% | 63% |
| | | Hf(OTf)₄ (2.6 g) | | |
| | | 200° C., 15 h, 40 bar H₂ | | |
| | | substrate to solvent volume | | |
| | | ratio = 0.075 | | |
| 9.2 | 30 g | Pd/C (10 wt. % Pd) (5.8 g), | 33% | 42% |
| | | Hf(OTf)₄ (2.6 g) | | |
| | | 200° C., 15 h, 40 bar H₂ | | |
| | | substrate to solvent volume | | |
| | | ratio = 0.063 | | |

Example 10: The C26 Product—Synthesis of 7-(4-Methylpentyl)-6-pentylpentadecane (RKBA-26) by Catalytic Condensation of 2MF with Trans-2-undecenal and Catalytic Hydrodeoxygenation of RKuns at-26

Molecular Formula of RKBA-26: C₂₆H₃₆O₃

### Step 1: Synthesis of RKunsat-26

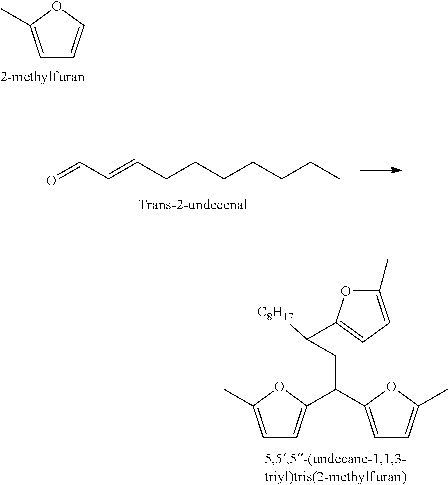

### Step 2: Synthesis of RKBA-26

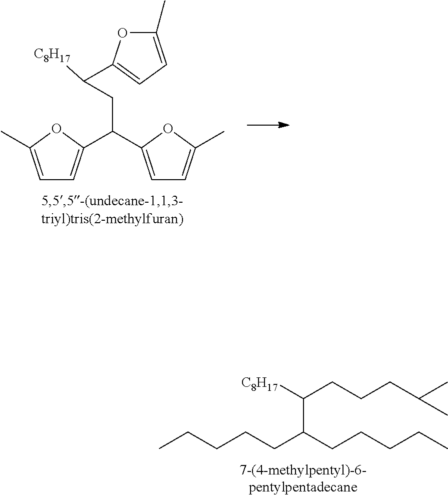

The RKBA-26 production involved two steps. The first step involved condensation of 2MF with trans-2-undecenal in the presence of an acid catalyst (e.g., P—SiO₂, Aquivion® PW98, Amberlyst®-15) in the temperature range of 60° C. to 80° C., which produced RKunsat-26 as the major product along with a C₂₁-product and a C₁₆-product (Table 10). After the reaction, the product mixture was filtered to separate out the catalyst, and was vacuum-distilled to remove any unconverted 2MF and water co-product.

The second step involved catalytic hydrodeoxygenation of RKunsat-26 product mixture of the first step with a physically mixed Pd/C and Hf(OTf)₄ catalyst in cyclohexane solvent using a Parr reactor (Table 11). The colorless product mixture was filtered from the catalyst, and vacuum distilled to separate the solvent. The colorless crude product's key specifications are shown in Table 12.

TABLE 10
Production of RKunsat-26 from Condensation
of 2-Methylfuran and Trans-2-Undecenal

| | | | | | % Yield | |
|---|---|---|---|---|---|---|
| | | | | | Products | |
| | | | | | of Carbon | |
| | Reaction | | Catalyst and | % Yield | No. 16-21 | % Total |
| No. | Scale | | Reaction Conditions | RKunsat-26 | (%)* | Yield |
| 10.1^{a} | 2 | g | P—SiO₂ | 79 | 20 | 99 |
| | | | 80° C.; 2 h | | | |
| 10.2^{a} | 2 | g | P—SiO₂ | 45 | 54 | 100 |
| | | | 25° C.; 2 h | | | |
| 10.3^{b} | 50 | g | P—SiO₂ | 70 | 29 | 99 |
| | | | 80° C.; 2 h | | | |
| 10.4^{b}** | 50 | g | P—SiO₂ | 71 | 27 | 98 |
| | | | 80° C.; 2 h | | | |
| 10.5^{b} | 50 | g | Amberlyst ®-15 | 56 | 42 | 98 |
| | | | 80° C.; 2 h | | | |
| 10.6^{c} | 200 | g | P—SiO₂ | 68 | 31 | 99 |
| | | | 80° C.; 2 h | | | |
| 10.7^{c} | 200 | g | P—SiO₂ | 66 | 33 | 99 |
| | | | 70° C.; 2 h | | | |

TABLE 11
Production of RKBA-26 from Hydrodeoxygenation of RKunsat-26

| | Reaction | | |
|---|---|---|---|
| | Scale Per | Catalyst and | % Total |
| No. | Batch | Reaction Conditions | Yield |
| 11.1 | 60 g | Pd/C (10 wt. % Pd) (6 g), | 94% |
| | | Hf(OTf)₄ (2 g) | |
| | | 200° C., 12 h, 40 bar H₂ | |
| | | substrate to solvent volume | |
| | | ratio = 0.12 | |
| 11.2 | 50 g | Pd/C (10 wt. % Pd) (6 g), | 95% |
| | | Hf(OTf)₄ (2g) | |
| | | 200° C., 12 h, 40 bar H₂ | |
| | | substrate to solvent volume | |
| | | ratio = 0.1 | |

TABLE 12
Key Specifications of RKBA-26

| | | | | Pour | |
|---|---|---|---|---|---|
| | KV40 | KV100 | | Point | Evaporation |
| Product | (cSt) | (cSt) | VI | (° C.) | Loss (%) |
| RKBA-26 | 31.1 | 4.9 | 70 | −51 | 3.04 |

The kinematic viscosity KV100 and KV40 were measured by ASTM D445. Viscosity was calculated from KV100 and KV40 (ASTM D2270). The pour point was measured by ASTM D97. The evaporation loss was determined by ASTM D 972 (6.5 hours at 100° C.).

Example 11: Cosmetic Formulations/Precursors From RKsat-17, RKsat-15, RKsat-22, RKBA-17, and RKBA-22.

Different cosmetic compositions, namely, 50% zinc oxide dispersion, liquid blush (oil-based), gel-blush (water-based), sunscreen, oil-in-water lotion, and water-in-oil lotion, were formulated using one or more of the afore-synthesized compounds as emollients. The emollients were tested for their efficacy namely in dryness; volatility; after-use effect; stability; viscosity; pH; microscopy; and flash point. A full sensory profile of the cosmetic compositions was developed.

The key observations for one or more emollient compounds are:
1. the emollients were stable and compatible with cosmetic additives and packaging materials;
2. the emollients could easily form water-in-oil emulsion by facilitating the solubilization of simple emulsifiers; and
3. the hydrophilic-lipophilic balance (HLB) requirement for making oil-in-water emulsions via steric stabilization was 11.

The formulation procedure of each of these cosmetic compositions is exemplified below in Examples 11a-11f.

Example 11a: Cosmetic Composition for Facial Sun Defense Using RKBA-22

Water (52 wt. %), Zemea® (INCI name=propanediol; 8 wt. %) and Ronacare® magnesium sulfate (INCI name=magnesium sulfate; 0.8 wt. %) were pre-mixed to prepare Phase A. Similarly, RKBA22 (16 wt. %) and Silube® 316 (INCI name=TMP lauryl dimethicone; 3.2 wt. %) were pre-mixed to prepare Phase B. Phase A was slowly added to Phase B with a fast propeller-stirring resulting into a homogenized Phase AB. Then ZinClear® XP Powder (INCI name=zinc oxide; 20 wt. %) was added to Phase AB with a dispersion-blade mixing until the mixture was completely smooth. The formulation was prepared by mixing at room temperature, without requiring high mixing energy. The final sunscreen product containing 20 wt. % zinc oxide was transparent, and exhibited no greasy after-feel.

Example 11b: Cosmetic Composition of Water-in-Oil Lotion Using RKBA-22

Water (65 wt. %), Zemea® (INCI name=propanediol; 10 wt. %) and Ronacare® magnesium sulfate (INCI name=magnesium sulfate; 1 wt. %) were pre-mixed to form Phase A. Similarly, RKBA22 (20 wt. %) and Silube® 316 (INCI name=TMP lauryl dimethicone; 4 wt. %) were pre-mixed to form Phase B. Then Phase A was slowly added to Phase B with continuous mixing until a uniform and homogenized formulation mixture was formed. The formulation was prepared by mixing at room temperature, without requiring high mixing energy. This 80% internal phase water-in-oil lotion had medium-to-light after-feel. A sensory profile of formulated lotion is shown in Table A1 below.

TABLE A1
Sensory Profile of RKBA-22 Formulated Water-in-Oil Lotion

| | Appearance | | Pick-Up | | Rub-On | | | | After Feel | |
|---|---|---|---|---|---|---|---|---|---|---|
| | Glossie- | Firm- | Stringi- | Sticki- | Spread- | Slimi- | Greasi- | Absor- | Glossie- | Sticki- |
| | ness | ness | ness | ness | ability | ness | ness | bency | ness | ness |
| Formulator-1 | 4.5 | 4 | 4 | 3.5 | 3 | 4 | 2.5 | 1.5 | 4 | 3.5 |
| Formulator-2 | 4 | 4 | 3.5 | 3 | 2.5 | 4 | 2.5 | 1.5 | 4 | 3.5 |
| Average | 4.25 | 4 | 3.75 | 3.25 | 2.75 | 4 | 2.5 | 1.5 | 4 | 3.5 |

Example 11c: Cosmetic Composition of Oil-in-Water Lotion using RKBA-22

Water (63.6 wt. %) was poured in a container and was called Phase A. Phase B was prepared by mixing Zemea® (INCI name=propanediol; 10 wt. %), Keltrol® CG-SFT (INCI name=Xanthan Gum; 1 wt. %) and Texturlux® Stabil (INCI name=hydrolyzed corn starch hydroxyethyl ether; 0.4 wt. %). A 20 wt. % RKBA-22 emollient was poured into a separate container and was called Phase C. Phase D was prepared by mixing Poly Suga® Mulse D6 (INCI name=sorbitan oleate decylglucoside cross-polymer; 2 wt. %) and Syneth™ O13K (INCI name=polyglyceryl-10 oleate; 3 wt. %). After preparing four phases separately, Phase B was added to Phase A with mixing until a uniform mixture Phase AB was formed. Similarly, Phase D was added to Phase C with mixing until uniform mixture Phase CD was prepared. Finally, Phase CD was added to Phase AB with stirring until uniformity was achieved and a homogenized mixture was prepared. The formulation was prepared by mixing at room temperature, without requiring high mixing energy. The final lotion had a light after-use feeling and was prepared using 100% bio-based ingredients. Sensory profile of formulated lotion is shown in Table A2.

TABLE A2
Sensory Profile of RKBA-22 Formulated Oil-in-Water Lotion

| | Appearance | | Pick-Up | | Rub-On | | | | After Feel | |
|---|---|---|---|---|---|---|---|---|---|---|
| | Glossi- | Firm- | Stringi- | Sticki- | Spread- | Slimi- | Greasi- | Absor- | Glossi- | Sticki- |
| | ness | ness | ness | ness | ability | ness | ness | bency | ness | ness |
| Formulator-1 | 3.5 | 2.5 | 2 | 1.5 | 4.5 | 2.5 | 1.5 | 3 | 3 | 2 |
| Formulator-2 | 3 | 2.5 | 2 | 1.5 | 4.5 | 2.5 | 1.5 | 3 | 3 | 2 |
| Average | 3.25 | 2.5 | 2 | 1.5 | 4.5 | 2.5 | 1.5 | 3 | 3 | 2 |

Example 11d: Cosmetic Composition of Liquid Blush Using RKBA-22

This cosmetic product was prepared by mixing the materials summarized in Table A3 in the quantities provided. The procedure for preparing the liquid blush is shown in Table A4.

TABLE A3
Materials for Liquid-Blush Formulation

| [symbol] | [symbol] | [symbol] | [symbol] | [symbol] | [symbol] |
|---|---|---|---|---|---|
| A | [symbol] | Water | Water | | Solvent |
| B | 1 | [symbol] | [symbol] / [symbol] Copolymer | [symbol] | Dispersant |
| B | 1 | Span 20 | [symbol] | [symbol] | Emulsifier |
| C | 8 | Titanium dioxide | Titanium Dioxide | Color Techniques | Color |
| C | 1.2 | Yellow iron oxide | Iron Oxides | Color Techniques | Color |
| C | 1.05 | Red iron oxide | Iron Oxides | Color Techniques | Color |
| C | 0.17 | Black iron oxide | Iron Oxides | Color Techniques | Color |
| D | 5 | [symbol] | Propandiol | [symbol] | [symbol] |
| D | 0.3 | [symbol] | Xanthan Gum | [symbol] | [symbol] |
| E | 3 | [symbol] | [symbol] Searate (and) [symbol] | [symbol] | Emulsifier |
| E | 2 | [symbol] | [symbol] | [symbol] | Emolient |
| F | 20 | [symbol] | [symbol] | [symbol] | Emolient |
| G | 0.8 | [symbol] | Propanediol (and) | [symbol] | [symbol] |
| | | | [symbol] alcohol (and) | | |
| | | | [symbol] alcohol (and) | | |
| | | | [symbol] | | |

[symbol]

indicates data missing or illegible when filed

TABLE A4
Procedure for Making Liquid Blush

| | |
|---|---|
| 1 | [symbol] |
| 2 | [symbol] |
| 3 | Add C to A/B with [symbol] |
| 4 | Heat A/B/C to 80 C. while stiring with [symbol] |
| 5 | [symbol] A/B/C |
| 6 | Premix D and add to A/B/C while [symbol] temperature |
| 7 | Add E while maintaining heat until dissolved |
| 8 | Remove from head and continue stiring |
| 9 | When [symbol] reaches 60 C. add F |
| 10 | When [symbol] is below 45 C., add [symbol] |

[symbol]

indicates data missing or illegible when filed

Phases A-G were prepared by mixing the ingredients as provided in Table A3. Particularly, Phase B was premixed until it was uniform. Phase B was then mixed into Phase A. Phase C was added to Phase AB in presence of stirring with a propeller blade. The mixture ABC was heated to 80° C. accompanied with stirring until it was rendered uniform. The mixture ABC was then homogenized. Premix D was added to the ABC mixture while maintaining the temperature at 80° C. Phase E was subsequently added maintaining heat until it was dissolved in the mixture. The heating was removed but the stirring continued in the next step. When the batch reached 60° C., Phase F was added. In the next step, when the batch reached 45° C., Phase G was added.

The formulation was prepared by mixing at room temperature. The final product was a liquid blush is an innovative light-feeling oil-water liquid crystalline gel network system featuring 20% bio-based emollient and a novel aqueous dispersion technology.

Example 11e: Cosmetic Compositions of Transparent Blush using RKBA-22

The materials and the formulation procedure are shown in Tables A5 and A6, respectively.

TABLE A5
Materials for Transparent Blush Formulation

| [symbol] | [symbol] | [symbol] | [symbol] | [symbol] | [symbol] |
|---|---|---|---|---|---|
| A | [symbol] | Water | Water | | Solvent |
| A | 9 | [symbol] | [symbol] | [symbol] | [symbol] |
| A | 0.9 | [symbol] Magnesium Sulfate | Magnesium Sulfate | [symbol] / [symbol] | [symbol] |
| B | [symbol] | [symbol] | [symbol] | [symbol] | [symbol] |
| B | [symbol] | [symbol] | [symbol] | [symbol] | Emulsifier |
| C | 8 | Titanium Dioxide | Titanium Dioxide | Color Techniques | Color |
| C | 1.2 | Yellow Iron Oxide | Iron [symbol] | Color Techniques | Color |
| C | 1 | Red Iron Oxide | Iron [symbol] | Color Techniques | Color |
| C | 0.17 | Black Iron Oxide | Iron [symbol] | Color Techniques | Color |

[symbol]

indicates data missing or illegible when filed

TABLE A6
Procedure for making transparent blush

| | |
|---|---|
| 1 | Premix A and B [symbol] |
| 2 | Slowly add A to B with [symbol] stiring |
| | until uniform and glossy |
| 3 | Homogenize if not already glossy |
| 4 | Premix C then slowly add to A/B with a |
| | dispersion blade until uniform |

[symbol]

indicates data missing or illegible when filed

As shown in Table A5, Phases A-C were prepared with the listed ingredients. More specifically, Phases A and B were separately prepared. Phase A was slowly added to Phase B with a fast stirring with a propeller until the mixture was uniform and glossy in appearance. It was further homogenized to make it glossy. Phase 3 premix was slowly added to the Phase AB with a dispersion blade until the formulation was rendered unform. The formulation was prepared upon mixing at room temperature, and required minimum mixing energy and time. The final product was a medium-to-light feeling water-in-oil gel emulsion blush, with an easy application.

Example 11f: Cosmetic Composition of a Perfecting Primer using RKBA-22

The materials and the formulation procedure are shown in Tables A7 and A8, respectively.

TABLE A7
Materials for Perfecting Primer Formulation

| PHASE | % | [symbol] | [symbol] | SUPPLIER | FUNCTION |
|---|---|---|---|---|---|
| A | [symbol] | Water | Water | | Solvent |
| A | 9 | [symbol] | Propanediol | [symbol] | [symbol] |
| A | [symbol] | [symbol] Magnesium | Magnesium Sulfate | [symbol] / [symbol] | [symbol] |
| | | Sulfate | | | |
| B | 18 | [symbol] | [symbol] | [symbol] | [symbol] |
| B | [symbol] | [symbol] | [symbol] | [symbol] | Emulsifier |
| C | 10 | Titanium Dioxide | Titanium Dioxide | Color Techniques | Color |

[symbol]

indicates data missing or illegible when filed

TABLE A8
Procedure for Making a Perfecting Primer

| | |
|---|---|
| 1 | Premix A and B [symbol] |
| 2 | Slowly add A to B with far [symbol] propeller |
| | mixing until uniform and glossy |
| 3 | Homogenize briefly if not already glossy |
| 4 | Slowly add C to A/B with dispersion blade |
| | mixing until uniform and smooth |

[symbol]

indicates data missing or illegible when filed

As shown in Table, A7, Phases A-C were prepared with the listed ingredients. More specifically, Phases A and B were separately prepared. Phase A was slowly added to Phase B with a fast stirring with a propeller until the mixture was uniform and glossy in appearance. It was further homogenized to make it glossy. Phase C premix was slowly added to the Phase AB with a dispersion blade until the formulation was rendered unform. The formulation was fast upon mixing at room temperature, and required minimum mixing energy and time.

Example 12: Formulation of Cosmetic Compositions Using RKBA-17 Emollient

Water-in oil and oil-in-water lotions were formulated using RKBA-17. The formulation procedure and sensory profiles of each of these cosmetics are exemplified below in Examples 12a and 12b.

Example 12a: Formulation of Oil-in-Water Lotion Using RKBA-17

The materials necessary for the formulation are listed in Table 9. Phases A, B, C, and D were separately prepared. Phase B was added to Phase A with stirring until the mixture Phase AB was rendered uniform. Similarly, Phase D was added to Phase C with stirring until Phase CD was rendered uniform. Finally phase CD was added to AB with stirring until uniform and homogenized mixture was obtained.

TABLE A9
Materials for Oil-in-Water Lotion

| Phase | Wt. % | Tradename | INCI name | Function |
|---|---|---|---|---|
| A | 63.6 | Water | | Solvent |
| B | 10 | Zemea ® | Propanediol | Humectant |
| B | 1 | Keltrol ® CG-SFT | Xanthan Gum | Polymer |
| B | 0.4 | Texturlux ® stabil | Hydrolyzed corn | Polymer |
| | | | starch hydroxyethyl | |
| | | | ether | |
| C | 20 | RKBA-17 | | Emollient |
| D | 2 | Poly Suga ® Mulse D6 | Sorbitan oleate | Emollient |
| | | | decylglucoside | |
| | | | cross polymer | |
| D | 3 | Syneth ® O13 K | Polyglyceryl-10 oleate | Emollient |

The formulation was easily amenable to be formed into the lotion. The procedure was similar to the other compound of the present invention RKBA-22 in terms of cold-processing, and minimum mixing energy requirements. The sensory profile of formulated lotion is shown in Table A10.

TABLE A10
Sensory Profile of RKBA-17 Formulated Oil-in-Water Lotion

| | Appearance | | Pick-Up | | Rub-On | | | | After Feel | |
|---|---|---|---|---|---|---|---|---|---|---|
| | Glossi- | Firm- | Stringi- | Sticki- | Spread- | Slimi- | Greasi- | Absor- | Glossi- | Sticki- |
| | ness | ness | ness | ness | ability | ness | ness | bency | ness | ness |
| Formulator-1 | 4 | 3 | 2 | 1 | 5 | 2 | 1 | 3 | 4 | 1 |
| Formalator-2 | 3.5 | 3 | 2 | 1 | 5 | 2 | 1 | 3 | 4 | 1 |
| Average | 3.75 | 3 | 2 | 1 | 5 | 2 | 1 | 3 | 4 | 1 |

Example 12b: Formulation of Water-in-Oil Lotion Using RKBA-17

The materials necessary for the formulation are listed in Table A11. Phases A and B were separately prepared. Phase A was slowly added to Phase B with continuous stirring until the mixture was rendered uniform. The mixture was homogenized if it was not glossy.

TABLE A11
Materials for Water-in-Oil Lotion

| Phase | Wt. % | Tradename | INCI name | Function |
|---|---|---|---|---|
| A | 65 | Water | | Solvent |
| A | 10 | Zemea ® | Propanediol | Humectant |
| A | 1 | RonaCare ® Magnesium | Magnesium | Salt |
| | | Sulfate | sulfate | |
| B | 20 | RKBA17-SFD | | Emollient |
| B | 4 | Silube ® 316 | TMP lauryl | Polymer |
| | | | dimethicone | |

The formulation was easily amenable to preparing the lotion. The procedure was similar to RKBA-22 in terms of cold-processing, and minimum mixing energy requirements. The sensory profile of formulated lotion is shown in Table A12.

TABLE A12
Sensory Profile of RKBA-17 Formulated Water-in-Oil Lotion

| | Appearance | | Pick-Up | | Rub-On | | | | After Feel | |
|---|---|---|---|---|---|---|---|---|---|---|
| | Głossi- | Firm- | Stringi- | Sticki- | Spread- | Slimi- | Greasi- | Absor- | Glossi- | Sticki- |
| | ness | ness | ness | ness | ability | ness | ness | bency | ness | ness |
| Formulator-1 | 4.5 | 4 | 3.5 | 3 | 3 | 4 | 2.5 | 2 | 4 | 3 |
| Formulator-2 | 4 | 4 | 3.5 | 3 | 2.5 | 4 | 2.5 | 2 | 4 | 3 |
| Average | 4.25 | 4 | 3.5 | 3 | 2.75 | 4 | 2.5 | 2 | 4 | 3 |

Example 13: Formulation of Cosmetic Products using RKsat-17 Emollient

Water-in-oil and oil-in-water lotions were formulated using RKsat-17. The formulation of these two products using RKsat-17 emollient followed the same formulation procedures as RKBA-17 described in Examples 12a and 12b, supra. When compared with the formulation efficacy of RKBA-22, oil-in-water formulation using RKsat-17 was relatively difficult and the formulation required a longer time to homogenize. In case of oil-in water lotion formulation, the phases did not homogenize after a long time of mixing. However, for use purposes, these formulations simply need to be shaken vigorously prior to application for cosmetic purposes. The sensory profiles of water-in-oil and oil-in-water lotions are given in Tables A13 and A14.

TABLE A13
Sensory Profile of RKsat-17 Formulated Water-in-Oil Lotion

| | Appearance | | Pick-Up | | Rub-On | | | | After Feel | |
|---|---|---|---|---|---|---|---|---|---|---|
| | Glossi- | Firm- | Stringi- | Sticki- | Spread- | Slimi- | Greasi- | Absor- | Glossi- | Sticki- |
| | ness | ness | ness | ness | ability | ness | ness | bency | ness | ness |
| Formulator-1 | 3.5 | 3.5 | 3 | 2 | 3.5 | 3 | 2.5 | 3 | 3.5 | 1 |
| Formulator-2 | 3 | 3.5 | 3 | 2 | 3.5 | 3 | 2.5 | 3 | 4 | 1.5 |
| Average | 3.25 | 3.5 | 3 | 2 | 3.5 | 3 | 2.5 | 3 | 3.75 | 1.75 |

TABLE A14
Sensory Profile of RKsat-17 Formulated Oil-in-Water Lotion

| | Appearance | | Pick-Up | | Rub-On | | | | After Feel | |
|---|---|---|---|---|---|---|---|---|---|---|
| | Glossie- | Firm- | Stringi- | Sticki- | Spread- | Slimi- | Greasi- | Absor- | Glossi- | Sticki- |
| | ness | ness | ness | ness | ability | ness | ness | bency | ness | ness |
| Formulator-1 | 3 | 2 | 1 | 1 | 5 | 2 | 1 | 4 | 3 | 1 |
| Formulator-2 | 3.5 | 2 | 1 | 1 | 5 | 2 | 1 | 4 | 3 | 1 |
| Average | 3.25 | 2 | 1 | 1 | 5 | 2 | 1 | 4 | 3 | 1 |

Example 14: Formulation of Cosmetic Products Using RKsat-22 Emollient

Water-in oil and oil-in-water lotions were formulated using RKsat-22. The formulation of these two products using RKsat-22 emollient followed the same formulation procedures as RKBA-17 described in Examples 12a and 12b above. When compared with the formulation efficacy of RKBA-22, oil-in water formulation using RKsat-22 took a longer time to homogenize. In case of oil-in water lotion formulation, the phases did not homogenize after a long time of mixing. However, for use purposes, these formulations simply need to be shaken vigorously prior to application for cosmetic purposes. The sensory profiles of water-in-oil and oil-in-water lotions are given in Tables A15 and A16.

TABLE A15
Sensory Profile of RKsat-22 Formulated Water-in-Oil Lotion

| | Appearance | | Pick-Up | | Rub-On | | | | After Feel | |
|---|---|---|---|---|---|---|---|---|---|---|
| | Glossi- | Firm- | Stringi- | Sticki- | Spread- | Slimi- | Greasi- | Absor- | Glossi- | Sticki- |
| | ness | ness | ness | ness | ability | ness | ness | bency | ness | ness |
| Formulator-1 | 2 | 2 | 2 | 2.5 | 4 | 3 | 2 | 2.5 | 4 | 2 |
| Formulator-2 | 2 | 2 | 2 | 2.5 | 4 | 3 | 2 | 2.5 | 4 | 1.5 |
| Average | 2 | 2 | 2 | 2.5 | 4 | 3 | 2 | 2.5 | 4 | 1.75 |

TABLE A16
Sensory Profile of RKsat-22 Formulated Oil-in-Water Lotion

| | Appearance | | Pick-Up | | Rub-On | | | | After Feel | |
|---|---|---|---|---|---|---|---|---|---|---|
| | Glossi- | Firm- | Stringi- | Sticki- | Spread- | Slimi- | Greasi- | Absor- | Glossi- | Sticki- |
| | ness | ness | ness | ness | ability | ness | ness | bency | ness | ness |
| Formulator-1 | 2.5 | 2 | 1 | 1 | 5 | 2 | 1 | 4 | 2 | 1 |
| Formulator-2 | 3 | 2 | 1 | 1 | 5 | 2 | 1 | 4 | 3 | 1 |
| Average | 2.75 | 2 | 1 | 1 | 5 | 2 | 1 | 4 | 2.5 | 1 |

All the above formulations are suitable for sunscreen formulation, for example.

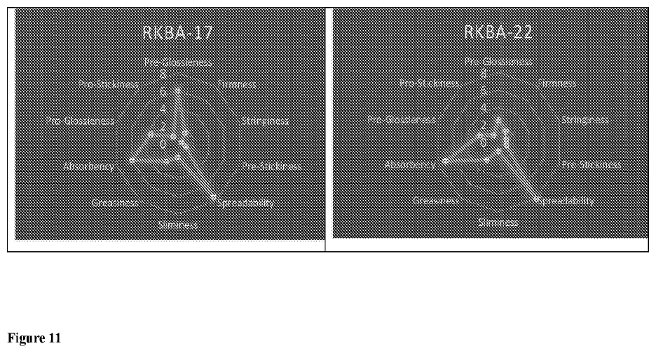

Example 15. Provided below is the sensory profile of neat RKBA-17 and RKBA-22 (also see FIG. 11):

| | Properties | | | | | | | | | |
|---|---|---|---|---|---|---|---|---|---|---|
| | Appearance | | Pick-Up | | Rub-On | | | | After Feel | |
| | Pre- | Firm- | Stringi- | Pre- | Spread- | Slimi- | Greasi- | Absor- | Pro- | Pro- |
| Compound | Glossiness | ness | ness | Stickiness | ability | ness | ness | bency | Glossiness | Stickiness |
| RKBA-17 | 6 | 1.5 | 0.5 | 1 | 7.5 | 1.5 | 2.5 | 6 | 3.5 | 1 |
| RKBA-22 | 2.5 | 1.5 | 1 | 1 | 8 | 1 | 2.5 | 7 | 2.5 | 1 |

1. A compound having the following formula;
(R₁−A)ₐ—CH(R₂)—CH₂—CH(R₃)(A−R1)_{b}
wherein:
(i) each of a is independently 1 or 2;
(ii) each of b is independently 0 or 1;
(ill) A is independently
(a) an unsaturated furan ring,
(b) a partially hydrogenated furan ring,
(c) a fully hydrogenated saturated furan ring,
(d) a saturated furan ring-opened moiety containing a hydroxyl group or a ketonic group, that is, —CH₂—CH₂—CH₂—CH₂—OH or —CH₂—CH₂—CH₂—CH═O,
(e) a partially saturated furan-ring opened moiety containing a hydroxyl group or a ketonic group, that is, —CH₂—CH₂—CH═CH—OH or —CH₂—CH═CH—CH═O where the position of the double bond is anywhere within the chain of the moiety, or
(f) —(CH₂)₄—;
(iv) R₁, R₂, and R₃ are independently selected from the group consisting of H, a furan ring, a tetrahydrofuran ring, and alkyl groups having carbon atoms of 1 to 18; and
(v) the total carbon content of the compound is in the range of 10 to 40.

2. The compound as recited in claim 1, wherein R₃ is a linear alkyl group.

3. The compound as recited in claim 1, the compound having one of the following structures: 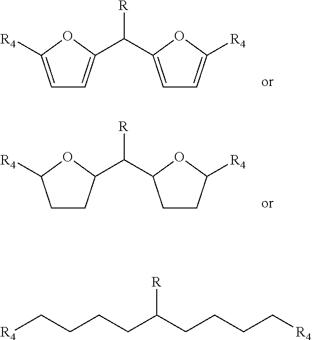
wherein R and R₄ are independently selected from the group consisting of H, a furan ring, a tetrahydrofuran ring, and alkyl groups having carbon atoms of 1 to 18.

4. The compound as recited in to claim 1, the compound having one of the following structures: 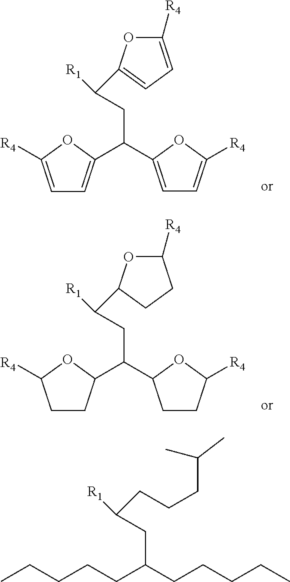
wherein R₁ and R₄ are independently selected from the group consisting of H, a furan ring, a tetrahydrofuran ring, and alkyl groups having carbon atoms of 1 to 18.

5. The compound as recited in claim 1, characterized by having a bio-based carbon content in the range of 30 to 100%, according to ASTM D6866 method.

6. A method of making a compound having the following formula:
(R₁−A)ₐ—CH(R₂)—CH₂—CH(R₃)(A−R1)_{b}
wherein:
each of a is independently 1 or 2;
(ii) each of b is independently 0 or 1;
(iii) A is independently
(a) an unsaturated furan ring,
(b) a partially hydrogenated furan ring,
(c) a fully hydrogenated saturated furan ring,
(d) a saturated furan ring-opened moiety containing a hydroxyl group o a ketonic group, that is, —CH₂—CH₂—CH₂—CH₂—OH or —CH₂—CH₂—CH₂—CH═O,
(e) a partially saturated furan-ring opened moiety containing a hydroxyl group or a ketonic group, that is, —CH₂—CH₂—CH═CH—OH or —CH₂—CH═CH—CH═O where the position of the double bond is anywhere within the chain of the moiety, or
(f) —(CH₂)₄—;
(iv) R₁, R₂, and R₃ are independently selected from the group consisting of H, a furan ring, a tetrahydrofuran ring, and alkyl groups having carbon atoms of 1 to 18; and
(v) the total carbon content of the compound is in the range of 1.0 to 40;
the method comprising:
(A) performing an add catalyzed condensation reaction of:
(I) a first component, comprising one or more of:
a 2-alkylfuran having the formula R₁-A, wherein R₁ is independently selected from the alkyl groups having carbon number of 1 to 18, and A is a furan ring,
with
(II) a second component, comprising one or more of:
(a) an aldehyde or a ketone having the formula of O═C(R₂)—R₅ wherein R₅ is independently selected from the alkyl groups having carbon number of 1 to 17, and R₂ is independently selected from the group consisting of H and alkyl groups having carbon number of 1 to 18,
(b) an aldehyde having the formula of O═CH—R wherein R is a furan ring or a tetrahydrofuran ring having carbon number of 4,
(c) an aldehyde or a ketone having the formula of O═C(R₂)—CH═CH—R₃ wherein R₂ is independently selected from the group consisting of H and alkyl groups having carbon number of 1 to 17, and R₃ is independently selected from the alkyl groups having carbon number of 1 to 17,
wherein at least one of the first component and the second component is at least partially bio-derived from a renewable carbon feedstock, and
wherein the catalyst comprises one acidic catalyst selected from the group consisting of organic liquid acids, inorganic liquid acids, solid Bronsted acids, and combinations thereof,
(B) selectively hydrogenating the condensation compound from Step (A) in the presence of a hydrogenation catalyst to obtain a saturated furan product, referred hereto hydrogenated saturated condensation compound or as RKsat-n,
(C) performing selective hydrodeoxygenation of:
(c1) the condensation compound in Step (A), or
(c2) the hydrogenated saturated condensation compound of Step (B),
in the presence of a hydrodeoxygenation catalyst to obtain a compound referred hereto as RKBA-n,
wherein the hydrodeoxygenation catalyst comprises the catalyst consisting of metal selected from a hydrogenation metal or a base metal, an acid site selected from a Lewis acid or a Bronsted acid, or a mixture thereof.

7. The method as recited in claim 6, wherein the renewable carbon feedstock is selected from the group consisting of biomass, rapeseeds, palm kernel, natural oils, waste cooking oils, castor seed, corn grain, soya bean grain, hard wood, soft wood, algae, natural coconut oil, palm oil, rapeseed oil, vegetable oil, corn oil, peanut oil, olive oil, canola oil, and sunflower oil. any waste cooking oils of one or more natural cooking oils, animal fats, combinations thereof, and mixtures thereof.

8. The method as recited in claim 6, wherein R₃ is a linear alkyl group,

9. The method as recited in claim 6, the compound having one of the following structures: 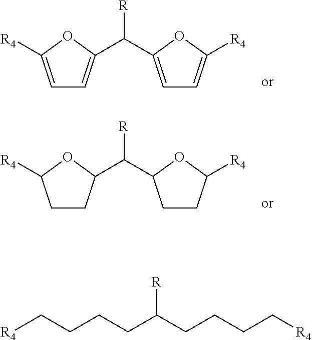
wherein R and R₄ are independently selected from the group consisting of H, a furan ring, a tetrahydrofuran ring, and alkyl groups having carbon atoms of 1 to 18.

10. The method as recited in claim 6, the compound having one of the following structures: 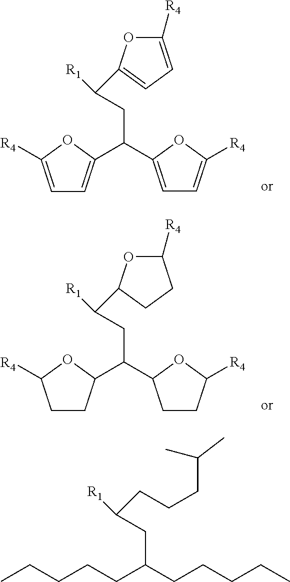
wherein R₁ and R₄ are independently selected from the group consisting of H, a furan ring, a tetrahydrofuran ring, and alkyl groups having carbon atoms of 1 to 18.

11. The method as recited in claim 6, wherein the step of providing an aldehyde comprises at least one of:
(i) dehydrogenating of one or more biomass derived alcohols,
(ii) selective hydrogenation of one or more carboxylic acids from one or more natural oils or waste cooking oils,
(iii) dehydration of biomass derived C₅ sugars, or
(iv) pyrolysis of castor seed.

12.-13. (canceled)

14. The method according to claim 6, wherein the acidic catalyst is selected from acidic resins, fluorinated resins, zeolites, phosphoric acid, phosphorous silica, orthophosphoric acid, HCl, H₂SO₄, methanesulfonic acid, p-toluene sulfonic acid, and combinations thereof.

15. The method according to claim 6:
wherein the hydrogenation catalyst comprises one metal catalyst selected from the group consisting of Ir, Ni, Pd, Pt, Ru, Mb, Zn, Ti, V, Cr, Mn, Fe, Co, and combinations thereof; and
wherein said hydrogenation catalyst is supported on a support material selected from the group consisting of activated carbon, porous carbon, silica, polymeric hybrid material, and weakly acidic material, mixture of Ni/NiO, Ni/C, Pd/C, Ru/C, Ni/SiO₂, Pd/SiO₂, Ni on zeolite supports, Ni/ZSM5, WR Grace-Raney Ni 2800, WR Grace-Raney Ni 3200, and a mixture of Ni/NiO with Al₂O₃, ZrO₂, Kieselguhr, Cr₂O₃ in different ratios.

16. (canceled)

17. The method according to claim 6, wherein the hydrodeoxygenation catalyst is:
(i) a bifunctional metal-acid catalyst co-existing with the metal and acid sites selected from the group consisting of Ni/ZSM5, Ni/zeolite, Ni/Al₂O₃, Raney Ni 2800, Raney Ni 3200, Pd/ZSM5, Pd zeolite, Pd/silica, Pd/Al₂O₃, a mixture of Ni/NiO with Al₂O₃, ZrO₂, Kieselguhr, Cr₂O with different ratios or a physically mixed catalyst consisting of a hydrogenation catalyst and an acid catalyst, or a supported metal-metal oxide catalyst of a general formula M¹MO wherein M¹=Ir, Ru, Ni, Co, Pd, or Rh and M=Re, Mo, W, Nb, Mn, V, Ce, Cr, Zn, Co, Y, or Al,
(ii) a physically-mixed catalyst comprising a hydrogenation catalyst and an acid catalyst, or
(iii) a supported metal-metal oxide catalyst of a general formula M¹MO,
wherein M¹−Ir, Ru, Ni, Co, Pd, or Rh; and
M=Re, Mo, W, Nb, Mn, V, Ce, Cr Zn, Co, Y, or Al.

18. A cosmetic composition, comprising:
(A) one or more compounds as recited in claim 1; and
(B) an effective amount of one or more additives selected from the group of pigment, fragrance, emulsifier, wetting agent, thickener, emollient, rheology modifier, viscosity modifier, gelling agent, antiperspirant agent, deodorant active, fatty acid salt, film former, anti-oxidant, humectant, opacifier, monohydric alcohol, polyhydric alcohol, fatty alcohol, preservative, pH modifier, a moisturizer, skin conditioner, stabilizing agent, proteins, skin lightening agents, skin-darkening agents, topical exfoliants, antioxidants, retinoids, refractive index enhancer, photostability enhancer, SPF improver, UV blocker, antibiotic agents, antiseptic agents, antifungal agents, anti-microbial agents, corticosteroid agents, anti-acne agents, and water,
wherein the one or more compounds have a branched molecular structure or a linear molecular structure, and
wherein the one or more compounds have an ether oxygen atom linkage or no oxygen atom.

19. The cosmetic composition as recited in claim 18, wherein said one or more compounds as recited in (A) comprise 0.1% to 99% by weight of the cosmetic composition.

20. The cosmetic composition as recited in claim 18, wherein the one or more compounds have a furan ring, a tetrahydrofuran ring, a partially hydrogenated furan ring, a partially saturated furan-ring opened moiety containing a hydroxyl or a ketonic group, that is, —CH₂—CH₂—CH═CH—OH or —CH₂—CH═CH—CH═O, where the position of the double bond is anywhere within the moiety, or —(CH₂)₄—.

21.-22. (canceled)

23. A skin care cosmetics composition, a decorative cosmetics composition, a hair-care cosmetic composition, or a personal care cosmetics composition comprising the cosmetic composition as recited in claim 18.

24. The cosmetic composition as recited in claim 18, for using as or in the preparation of, emollients, lotions, water-in-oil emulsions, oil-in-water emulsions, creams, gels, flexible solids, skin care cosmetics, moisturizing preparations, ointments, oils, cleansers, make-up removers, night and day treatments, skin reparatives, facial sun defense, sunscreens, blush, liquid blush, transparent blush, perfecting primer, liquid soaps, color foundations, make-ups, concealing sticks, rouge, tanning creams, eye shadow creams, shampoo, body wash, facial cleanser, face mask, bubble bath, intimate wash, bath oil, cleansing milk, micellar water, cleansing wipes, hair mask, perfume, liquid soap, shaving soap, shaving foam, cleansing foam, day cream, anti-ageing cream, body milk, body lotion, body mousse, face serum, eye cream, sunscreen lotion, sun cream, face cream, after-shave lotion, pre-shaving cream, depilatory cream, skin-whitening gel, self-tanning cream, anti-acne gel, mascara, foundation, primer, concealer, blush, bronzer, blemish balm (bb) cream, eyeliner, night cream, eye brow gel, highlighter, lip stain, hand sanitizer, hair oil, nail varnish remover, conditioner, hair styling gel, hair styling cream, anti-frizz serum, scalp treatment, hair colorant, split end fluid, deodorant, antiperspirant, baby cream, insect repellent, hand cream, sunscreen gel, foot cream, exfoliator, body scrub, cellulite treatment, bar soap, cuticle cream, lip balm, hair treatment, eye shadow, bath additive, body mist, eau de toilette, mouthwash, toothpaste, lubricating gel, moisturizer, serum, toner, aqua sorbet, cream gel, styling mousse, dry shampoo, lip stick, lip gloss, hydro-alcoholic gel, body oil, shower milk, illuminator, lip crayon, hair spray, combing cream, sunblock, cosmetic product that colors the skin, cosmetic product that lightens the skin, cosmetic products that repairs the skin, cosmetic product that moisturizes the skin, cosmetic product that smooth the skin, cosmetic product that conditions the skin, cosmetic products that protects the skin, cosmetic product that deans the skin, cosmetic product that rejuvenates the skin, cosmetic product that prevents the loss of moisture, and cosmetic product that reverses damages of the skin.

25. A process for preparing the cosmetic composition as recited in claim 18;
said process comprising the steps of:
(I) providing:
(A) at least one compound having the following formula:
(R₁−A)ₐ—CH(R₂)—CH₂—CH(R₃)(A−R1)_{b}
wherein:
(i) each of a is independently 1 or 2;
(ii) each of b is independently 0 or 1;
(iii) A is independently
(a) an unsaturated furan ring,
(b) a partially hydrogenated furan ring,
(c) a fully hydrogenated saturated furan ring,
(d) a saturated furan ring-opened moiety containing a hydroxyl group or a ketonic group, that is, —CH₂—CH₂—CH₂—CH₂—OH or —CH₂—CH₂—CH₂—CH═O,
(e) a PARTIALLY saturated furan-ring opened moiety containing a hydroxyl group or a ketonic group, that is, —CH₂—CH₂—CH═CH—OH or —CH₂—CH═CH—CH═O where the position of the double bond is anywhere within the chain of the moiety, or
(f) —(CH₂)₄—;
(iv) R₁, R₂, and R₃ are independently selected from the group consisting of H, a furan ring, a tetrahydrofuran ring, and alkyl groups having carbon atoms of 1 to 18; and
(v) the total carbon content of the compound is in the range of 10 to 40, and
(B) an effective amount of one or more additives selected from the group consisting of pigment, fragrance, emulsifier, wetting agent, thickener, emollient, rheology modifier, viscosity modifier, gelling agent, antiperspirant agent, deodorant active, fatty acid salt, film former, anti-oxidant, humectant, opacifier, monohydric alcohol, polyhydric alcohol, fatty alcohol, preservative, pH modifier, a moisturizer, skin conditioner, stabilizing agent, proteins, skin lightening agents, topical exfoliants, antioxidants, retinoids, refractive index enhancer, photo-stability enhancer, SPF improver, UV blocker, antibiotic agents, antiseptic agents, antifungal agents, corticosteroid agents, anti-acne agents, water and mixtures thereof; and
(II) physically or chemically mixing or blending the ingredients in (A) and (B).
